(19) United States
(12) Patent Application Publication
(10) Pub. No.: US 2014/0141469 A1
(43) Pub. Date: May 22, 2014

(54) METHODS OF PRODUCING HUMAN HEMOGLOBIN IN A BIOREACTOR

(71) Applicants: Douglas P. Henderson, Odessa, TX (US); Bryan Jacob Zachary Smith, Odessa, TX (US)

(72) Inventors: Douglas P. Henderson, Odessa, TX (US); Bryan Jacob Zachary Smith, Odessa, TX (US)

(21) Appl. No.: 14/164,738

(22) Filed: Jan. 27, 2014

Related U.S. Application Data
(63) Continuation of application No. PCT/US2012/048654, filed on Jul. 27, 2012.
(60) Provisional application No. 61/512,770, filed on Jul. 28, 2011.
(60) Provisional application No. 61/553,679, filed on Oct. 31, 2011.

(51) Int. Cl. C07K 14/805; C12R 1/19

(52) U.S. Cl. CPC C07K 14/805; C12R 1/19
USPC 435/69.6

(57) ABSTRACT

Methods for producing hemoglobin in a bioreactor comprising providing a bioreactor containing a strain of E. coli comprising hemoglobin producing genes and heme transport genes on a one plasmid system; introducing a carbon source into the bioreactor; growing a culture of the E. coli strain; inducing the hemoglobin producing genes; and allowing the E. coli to produce hemoglobin.

### CROSS-REFERENCE TO RELATED APPLICATIONS

This application is a continuation application of International Application No. PCT/US2012/048654 filed Jul. 27, 2012; which claims the benefit of U.S. Provisional Patent Application Ser. No. 61/512,770, filed on Jul. 28, 2011, and U.S. Provisional Patent Application Ser. No. 61/553,679, filed on Oct. 31, 2011, the entire disclosures of which are hereby incorporated by reference.

### BACKGROUND

A number of attempts have been made to find alternatives to human blood for patients who require transfusions. The use of blood transfusions has always come with certain risks and drawbacks. When someone receives a blood transfusion, they are potentially at risk of contracting a disease or of having a fatal hemolytic reaction. Blood transfusions also require blood typing of the recipient prior to use, thus lengthening the time required to provide a patient with emergency care. Donated blood also has a limited supply and a short shelf life.

The availability of a majority of blood substitutes depends on outdated blood or blood collected for the purpose of hemoglobin extraction. This can pose potential problems if a large enough crisis or contamination of the blood supply occurs. In order to produce a blood substitute not limited by the availability of blood as a starting material, some researchers have focused on the use of recombinant DNA technology. Using recombinant hemoglobin has an additional advantage in that the hemoglobin genes can be modified so that versions of the hemoglobin protein can be made that have better properties than hemoglobin found in red blood cells. Many organisms have been used for the production of recombinant human hemoglobin, but Escherichia coli has emerged as the most promising organism for this purpose. U.S. Patent Application Publication No. 20070166792, the entirety of which is hereby incorporated by reference, describes methods of coexpressing Plesiomonas shigelloides heme transport system genes and the human hemoglobin genes in E. coli.

Small scale (5 mL) human hemoglobin production assays have previously been developed. In these assays, E. coli BL21(DE3) coexpressing the Plesiomonas shigelloides heme transport system genes and the human hemoglobin genes have been grown in heme-supplemented media to produce high levels of human hemoglobin. The heme transport genes allow high levels of heme to be moved into the cell so more hemoglobin can be made. However, the scale of such an operation is not economical and an economical method of producing recombinant human hemoglobin for use as a blood substitute is highly desirable.

### SUMMARY

The present disclosure generally relates methods of producing hemoglobin in a bioreactor. More particularly, the present disclosure generally relates to the development of a method to produce hemoglobin in a bioreactor culture of Escherichia coli BL21(DE3) transformed with a plasmid containing Plesiomonas shigelloides heme transport genes and modified human hemoglobin genes.

In one embodiment, the present disclosure provides a method for producing hemoglobin in a bioreactor comprising: providing a bioreactor containing a strain of E. coli comprising hemoglobin producing genes and heme transport genes on a one plasmid system; introducing a carbon source into the bioreactor; growing a culture of the E. coli strain; inducing the hemoglobin producing genes; and allowing the E. coli to produce hemoglobin.

In another embodiment, the present disclosure provides a method for producing hemoglobin in a bioreactor comprising: providing a bioreactor containing a strain of E. coli comprising hemoglobin producing genes and heme transport genes on a one plasmid system; introducing oxygen, base, acid, and glucose feeds into the bioreactor; introducing a phosphate feed into the bioreactor; growing a culture of the E. coli strain; inducing the hemoglobin producing genes; and allowing the E. coli to produce hemoglobin.

In another embodiment, the present disclosure provides a method for producing hemoglobin in a bioreactor comprising: providing a bioreactor containing a strain of E. coli comprising hemoglobin producing genes and heme transport genes on a one plasmid system; introducing oxygen, base, acid, and glucose feeds into the bioreactor; introducing a phosphate feed into the bioreactor; growing a culture of the E. coli strain; inducing the hemoglobin producing genes; and allowing the E. coli to produce hemoglobin, wherein the feeds are introduced into the bioreactor at proper levels and at proper times that allow for optimal growth and hemoglobin production.

The features and advantages of the present invention will be apparent to those skilled in the art. While numerous changes may be made by those skilled in the art, such changes are within the spirit of the invention.

### DRAWINGS

Some specific example embodiments of the disclosure may be understood by referring, in part, to the following description and the accompanying drawings.

FIG. 1 is a drawing of a bioreactor vessel.

FIG. 2 is a chart depicting a flow rate standard curve.

FIG. 3 is a genetic and partial restriction enzyme map.

FIG. 4 is a chart depicting the Batch/Constant-rate glucose feed.

FIG. 5 is a chart depicting a modified version of stepwise glucose feed.

FIG. 6 is a chart depicting the first mass flow equation based glucose feed.

FIG. 7 is a chart depicting a comparison between the first and second mass flow equation based glucose feeds.

FIG. 8 is a pH graph.

FIG. 9 is a chart depicting a comparison between the second mass flow equation based glucose feed and the glucose feed of the first phosphate feed run.

FIG. 10 is an agitation graph.

FIG. 11 is a photo depicting the relationship of plasmid loss to hemoglobin production.

FIG. 12 is a chart depicting a comparison between the glucose feed of the 1.5 mg/mL heme feed runs and the new higher flow rate glucose feed.

FIG. 13 is an agitation graph.

FIG. 14 is a process flow diagram.

FIG. 15 is a process flow diagram.

FIG. 16 is a photo of a culture.

FIG. 17 is a graph depicting the flow rate of glucose and phosphate over time.

FIG. 18 is a graph depicting glucose concentration and dry cell weight over time.

FIG. 19A is a graph depicting hemoglobin production over time.

FIG. 19B is an immunoblot depicting hemoglobin produced.

The patent or application file contains at least one drawing executed in color. Copies of this patent or patent application publication with color drawing(s) will be provided by the Office upon request and payment of the necessary fee.

### DESCRIPTION

The present disclosure generally relates methods of producing hemoglobin in a bioreactor. More particularly, the present disclosure generally relates to the development of a method to produce hemoglobin in a bioreactor culture of Escherichia coli BL21(DE3) transformed with a plasmid containing Plesiomonas shigelloides heme transport genes and modified human hemoglobin genes.

The present disclosure provides a demonstration that it is possible to scale up human hemoglobin production from cultures grown in test tubes to large scale (multiple liters) human hemoglobin production with cultures grown in a bioreactor. By using a bioreactor, cultures were grown that reached cell densities 66 to 74 times the cell density of test tube cultures. With these high cell density cultures, the production rate of hemoglobin per mL was 25 times more than test tube cultures.

In one embodiment, the present disclosure provides a method for producing hemoglobin in a bioreactor comprising: providing a bioreactor containing a strain of E. coli comprising hemoglobin producing genes and heme transport genes on a one plasmid system, introducing oxygen, base, acid, and glucose feeds into the bioreactor, introducing a phosphate feed into the bioreactor, introducing a heme/base feed into the bioreactor, inducing the hemoglobin producing genes; and allowing the E. coli to produce hemoglobin.

In another embodiment, the present disclosure provides a method for producing hemoglobin in a bioreactor comprising: providing a bioreactor containing a strain of E. coli comprising hemoglobin producing genes and heme transport genes on a one plasmid system, introducing oxygen, base, acid, and glucose feeds into the bioreactor, introducing a phosphate feed into the bioreactor, introducing a heme/base feed into the bioreactor, inducing the hemoglobin producing genes; and allowing the E. coli to produce hemoglobin, wherein the feeds are introduced into the bioreactor at proper levels and at proper times that allow for optimal growth and hemoglobin production.

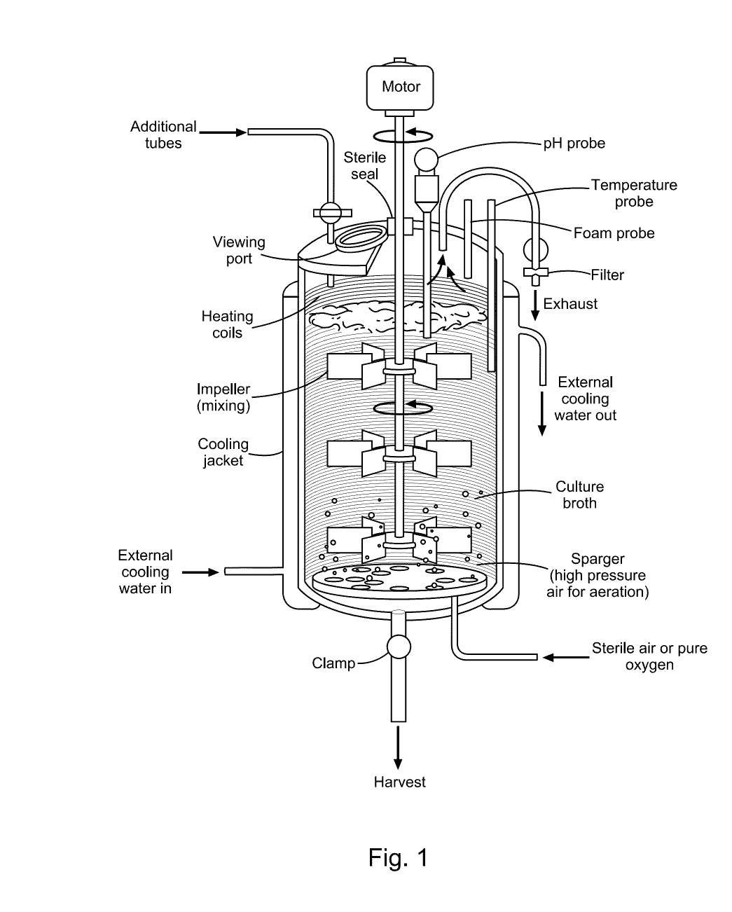

Referring now to FIG. 1, FIG. 1 shows a representative bioreactor vessel. The pH monitoring probe feeds data to a computer controller that then activates either an acid or base pump to return the pH to a preset value. A dissolved oxygen probe (not pictured) sends data to a computer controller that then maintains the dissolved oxygen at a set level by controlling the agitation rate of the impellers, increasing the gas flow rate, switching from ambient air to a pure oxygen tank, or using a combination of all three. The sparger causes gas entering the vessel to be released as small bubbles allowing for better diffusion of oxygen into the media. The impeller helps to break these bubbles down to a smaller size and distribute any fluids added to the bioreactor. Nutrients flow into the bioreactor through an addition tube. Addition of the nutrients is controlled by a computer program which alters the flow rate of attached pumps based on the amount of time the culture has grown. A computer controller uses data from the temperature probe to maintain a set temperature through the use of the cooling water jacket and the heating coils. A foam probe prevents foaming by triggering the addition of anti-foam if foam is detected.

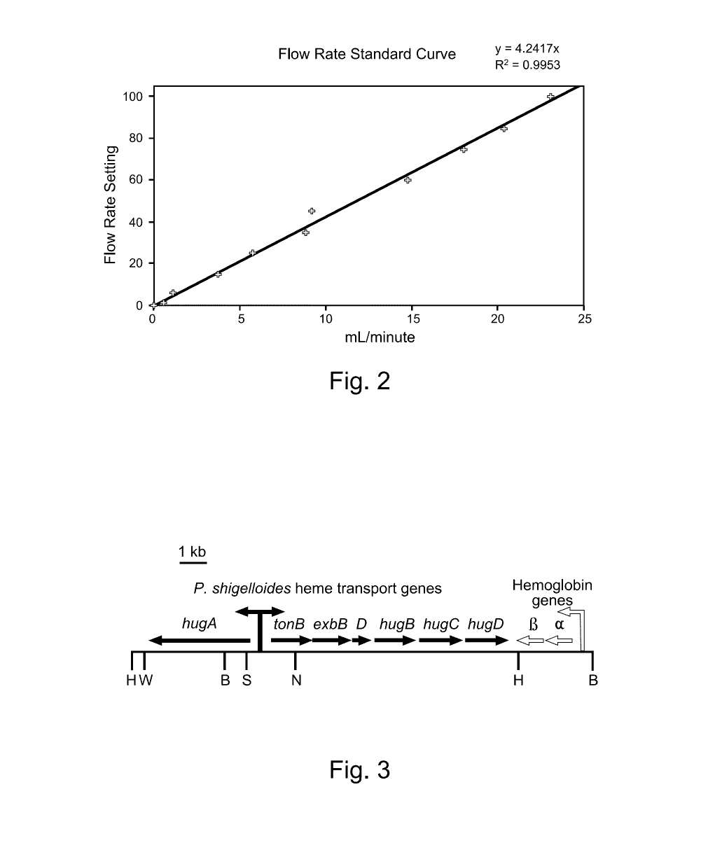

Referring now to FIG. 2, FIG. 2 shows a chart depicting a flow rate standard curve. Pumps used with bioreactors should release a linearly increasing amount of volume as the flow rate is increased. An R2 function should be used to ensure the data points collected for the pump are valid. The R2 value represents how close the data points are to the best fit linear line. If R2=1, the data points fall in a perfectly linear line. An R2 value that is 0.99 or better is acceptable.

Referring now to FIG. 3, FIG. 3 shows a genetic and partial restriction enzyme map of the Plesiomonas shigelloides heme transport system genes and the triple mutant human hemoglobin genes on pHb0.0TM 1hug. Vertical bent arrows represent promoters and horizontal arrows represent genes. The promoter and genes of the P. shigelloides heme transport system are shown in green and the promoter and genes of the triple mutant version of human hemoglobin genes (TM1) are shown in red. Restriction enzyme sites are represented by vertical lines along the bottom: B, BamHI; H, HindIII; N, NheI; S, SalI; and W, BsiWI.

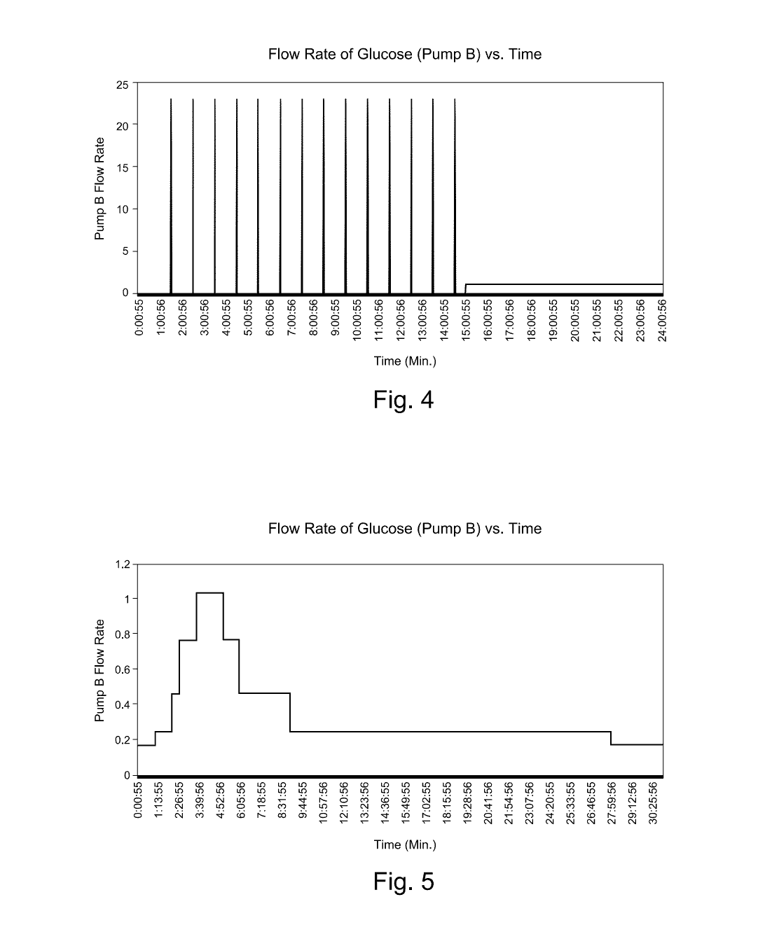

Referring now to FIG. 4, FIG. 4 shows a chart depicting a batch/constant-rate glucose feed. In the batch addition technique used during the first part of the run large quantities of glucose were added in a short period of time at regularly spaced intervals. During the intervals between glucose additions the bacteria reduce the glucose concentration in the culture. The last part of the glucose feed is a constant-rate section which is being done in anticipation of an eventual hemoglobin induction period. It was assumed the culture would have a decreased growth rate and a continuous demand for glucose as a result of hemoglobin production.

Referring now to FIG. 5, FIG. 5 shows a chart depicting a modified version of stepwise glucose feed. This glucose feed is designed to increase the amount of glucose added as the number of cells in the bioreactor increases and decrease the amount of glucose added after the growth rate of the culture begins to slow down.

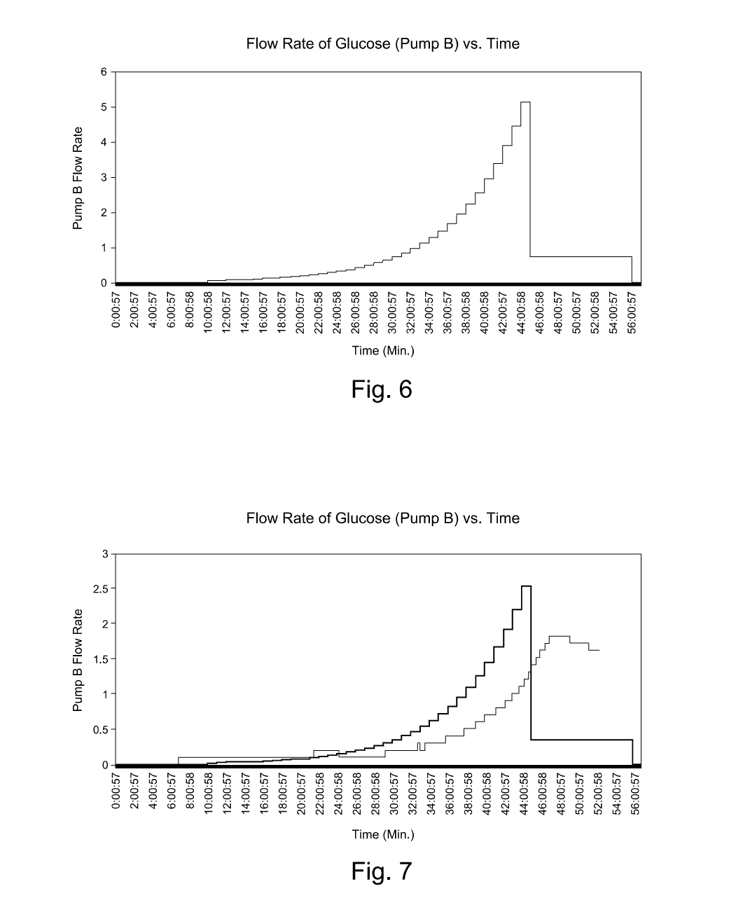

Referring now to FIG. 6, FIG. 6 shows a chart depicting the first mass flow equation based glucose feed. The first mass flow equation based glucose feed was based on a prior described mass flow equation and a 40% glucose feed solution. A mass flow equation is used to predict the rate at which glucose addition should increase to meet the demands of an ever increasing bacterial population. The low constant flow rate from 45 to 56 hours was designed for use during an eventual 11 hour hemoglobin induction period.

Referring now to FIG. 7, FIG. 7 shows a comparison between the first and second mass flow equation based glucose feeds. The red line represents the flow rate values for the first mass flow equation based nutrient feed (FIG. 6) that have been converted from 40% glucose feed values to 79.5% glucose feed values so that a direct comparison between the first (FIG. 6) and second (yellow line) mass flow equation based feeds is possible. After using a 40% glucose feed, a 79.5% glucose feed was used and a modified version of the first mass flow equation based nutrient feed (yellow line). The glucose flow rate of the first mass flow equation based feed (red line) was reduced in the second mass flow equation based feed (yellow line) to maintain the glucose concentration around 0.05%. The decrease in flow rate at the end of the run was gradual and designed for an expected decrease in glucose demand when the bacteria reached their stationary growth phase (the reduced growth phase that occurs at the end of a culture's growth).

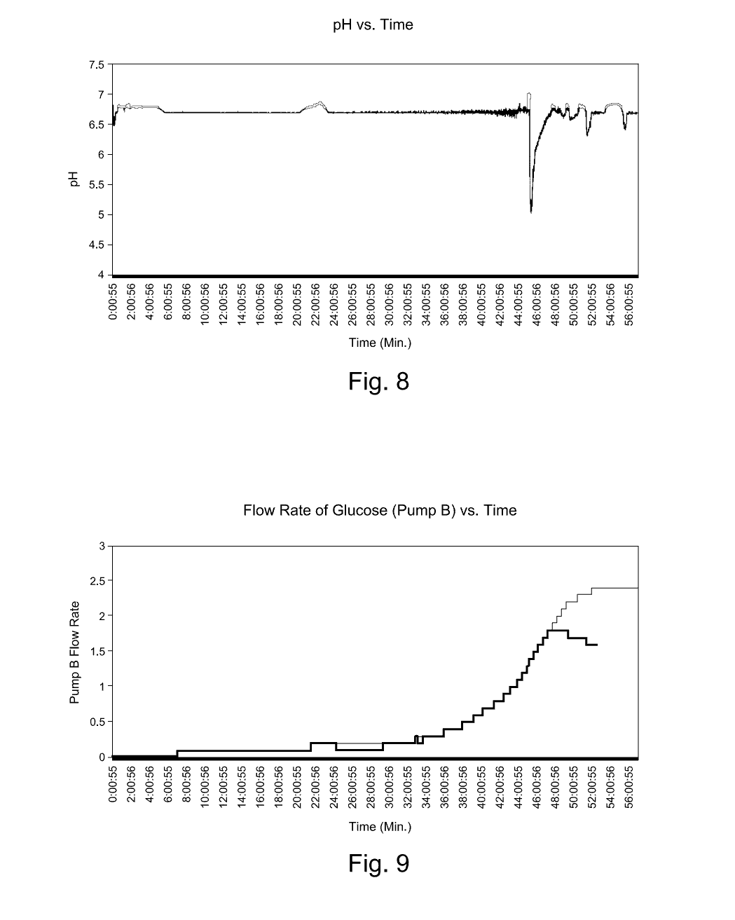

Referring now to FIG. 8, FIG. 8 shows a pH graph for the first 0.97% yeast extract run. During the first 0.97% yeast extract run (255 OD600 run), large quantities of phosphoric acid were added to the culture as a result of an improperly setup pH control program. Periods of phosphoric acid addition are highlighted in red.

Referring now to FIG. 9, FIG. 9 shows a comparison between the second mass flow equation based glucose feed and the glucose feed of the first phosphate feed run. The yellow line represents the flow rate values for the second mass flow equation based feed (FIG. 7 yellow line). The purple line represents where the flow rate values for the glucose feed of the first phosphate feed run differ from the flow rate values of the second mass flow equation based feed (yellow).

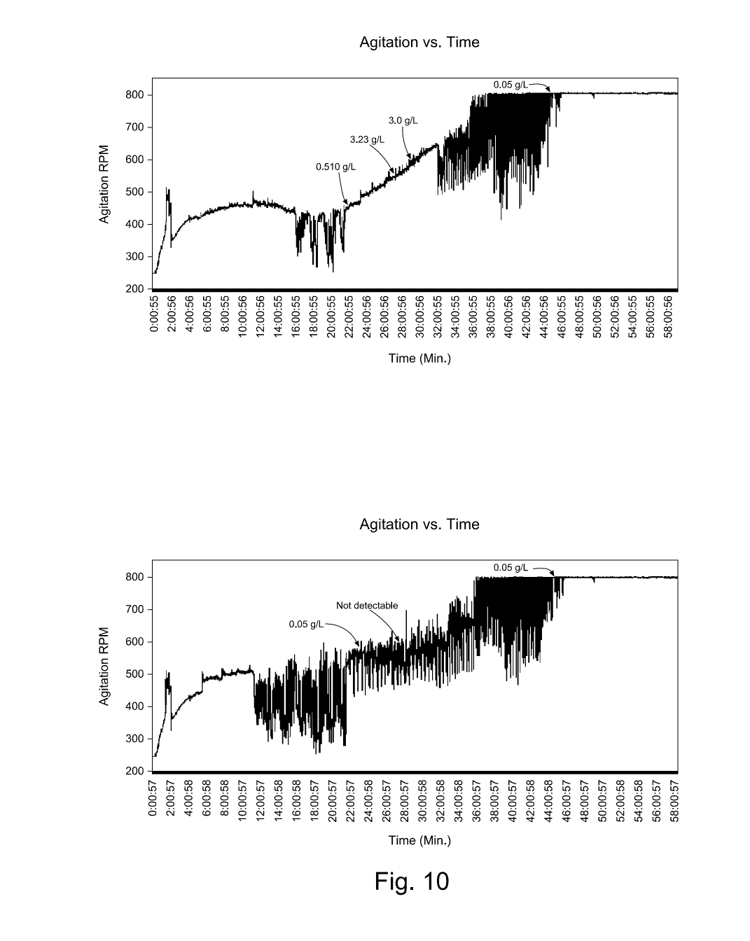

Referring now to FIG. 10, FIG. 10 shows how agitation graphs predict plasmid loss. The yellow numbers in the above figures indicate the glucose concentration of the culture at that time point. (A) Agitation graph of a hemoglobin producing (red) run. When the glucose concentration is above 0.05% the agitation graph is smooth and the plasmid is retained. (B) Agitation graph of a run where little hemoglobin was produced (brown run). When the glucose concentration remains below 0.05% the agitation graph is noisy and the plasmid is lost.

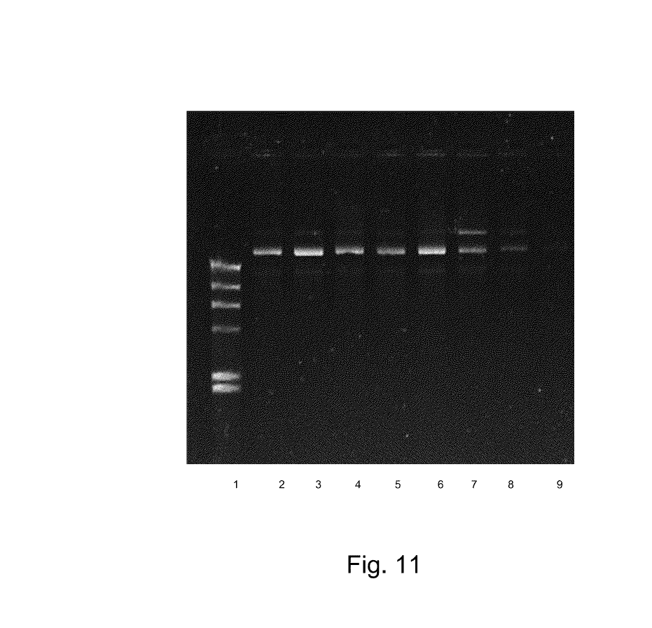

Referring now to FIG. 11, FIG. 11 shows the relationship of plasmid loss to hemoglobin production. Lanes 2 through 9 contain plasmid isolations prepared from the same quantity of cells. Lane 1, λ HindIII molecular weight marker; Lane 2, inoculum control; Lane 3, red run 29.25 hours; Lane 4, red run 45.08 hours; Lane 5, red run 58.43 hours; Lane 6, inoculum control; Lane 7, brown run 27.75 hours; Lane 8, brown run 44.5 hours; Lane 9, brown run 58.03 hours. The plasmid isolations in lanes 3 through 5 are from samples taken during the red run shown in FIG. 10A. Lanes 7 through 9 are plasmid isolations of samples taken from the brown run shown in FIG. 10B. Lanes 2 and 9 contain plasmid isolations from a standard inoculum culture. A plasmid isolation from a representative inoculum culture was used to compare the plasmid concentration of bioreactor samples to the plasmid concentration of a typical inoculum culture. The culture that produced hemoglobin (FIG. 10A) maintained a high plasmid concentration (lanes 3-5). However, the plasmid concentration decreased drastically (lanes 7-9) during the run of the culture where minimal hemoglobin was produced (FIG. 10b).

Referring now to FIG. 12, FIG. 12 shows a comparison between the glucose feed of the 1.5 mg/mL heme feed runs and the 1.5 mg/mL heme feed run with the new higher flow rate glucose feed. The pink line represents the glucose feed of the 1.5 mg/mL heme feed runs where the variable growth rates of different cultures was not taken into account. If a culture grew faster than expected, glucose starvation and subsequent plasmid loss occurred. To account for the variable growth rates of different cultures and prevent plasmid loss, a new higher flow rate glucose feed (orange line) was developed where the glucose concentration was kept above 0.2% (2.0 g/L) throughout most of the run. Through the use of the new higher flow rate glucose feed plasmid loss from glucose starvation was prevented.

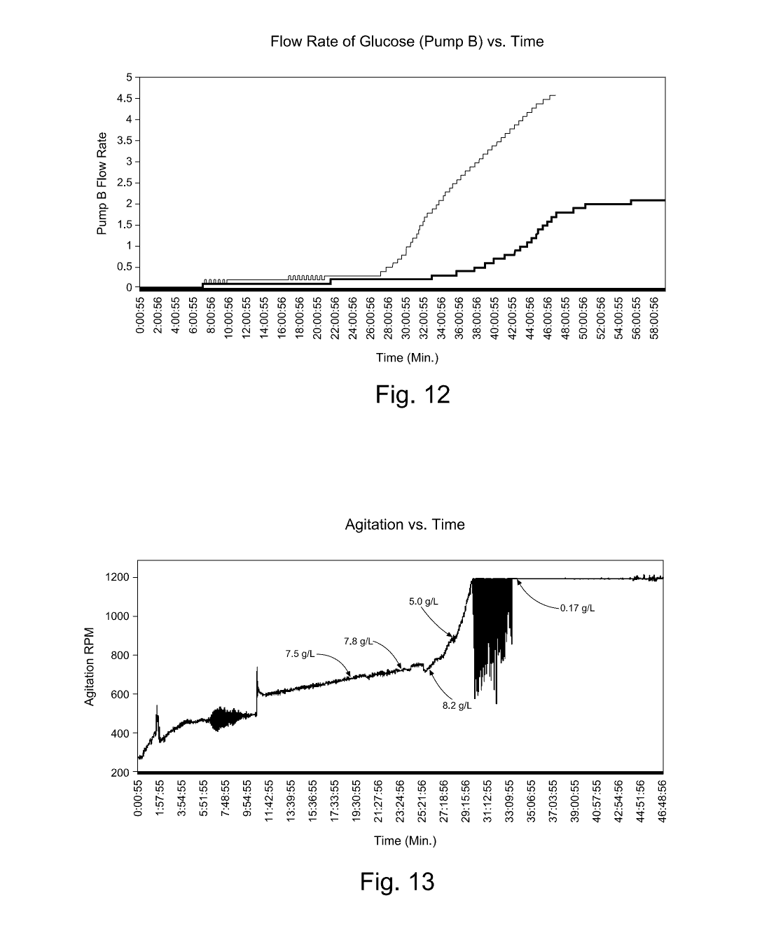

Referring now to FIG. 13, FIG. 13 shows an agitation graph of a culture grown with the new higher flow rate glucose feed. By using the new higher flow rate glucose feed (FIG. 12 orange line) to grow bioreactor cultures the glucose concentration of the culture was maintained above 0.2% (2.0 g/L) for a majority of the run. This allowed the culture to maintain the plasmid at high concentration levels. With the new higher flow rate glucose feed (FIG. 12 orange line) cultures were grown that more consistently produced high amounts of hemoglobin. The agitation graph was very smooth; the noise associated with plasmid loss had disappeared. The section of the agitation graph between 29.25 and 33.17 hours is not a result of plasmid loss but instead is caused by pure oxygen being fed into the culture to maintain the dissolved oxygen at 40% when the maximum agitation (1200 rpm) is reached.

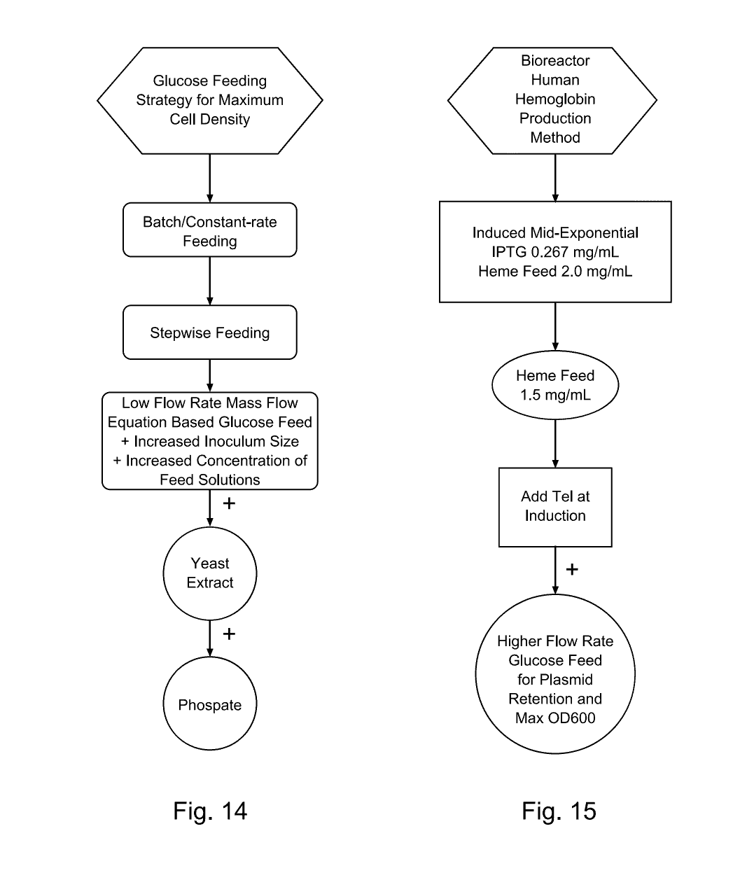

Referring now FIG. 14, FIG. 14 shows the development of the nutrient feeding strategy for production of high cell density cultures. Orange represents the goal of the development process. Blue indicates the failed glucose feeding strategies. Green indicates the components that make up the high cell density nutrient feeding strategy.

Referring now to FIG. 15, FIG. 15 shows the development of the bioreactor human hemoglobin production method. Orange represents the goal of the development process. Brown indicates minimal hemoglobin production. Red indicates hemoglobin production. The different shades of red indicate relative quantities of hemoglobin produced.

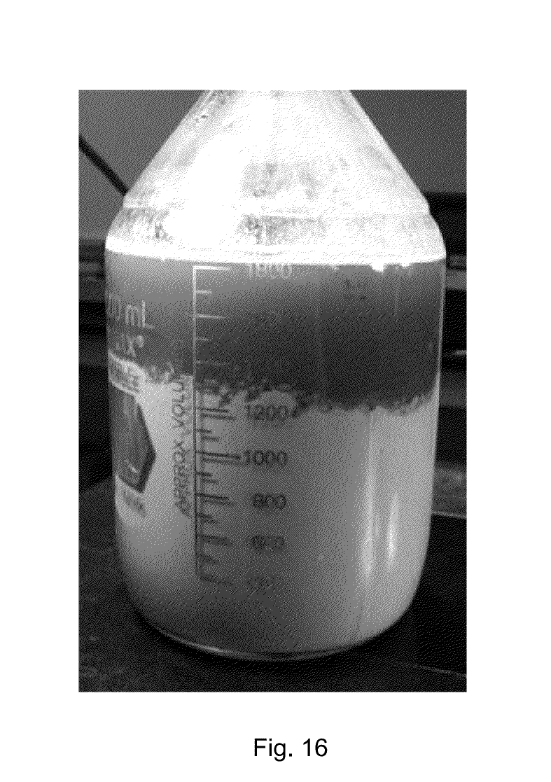

Referring now to FIG. 16, FIG. 16 shows an example of a culture that produced high amounts of human hemoglobin. This culture sat undisturbed for 1 week. The human hemoglobin present in the supernatant was released as a result of cell lysis.

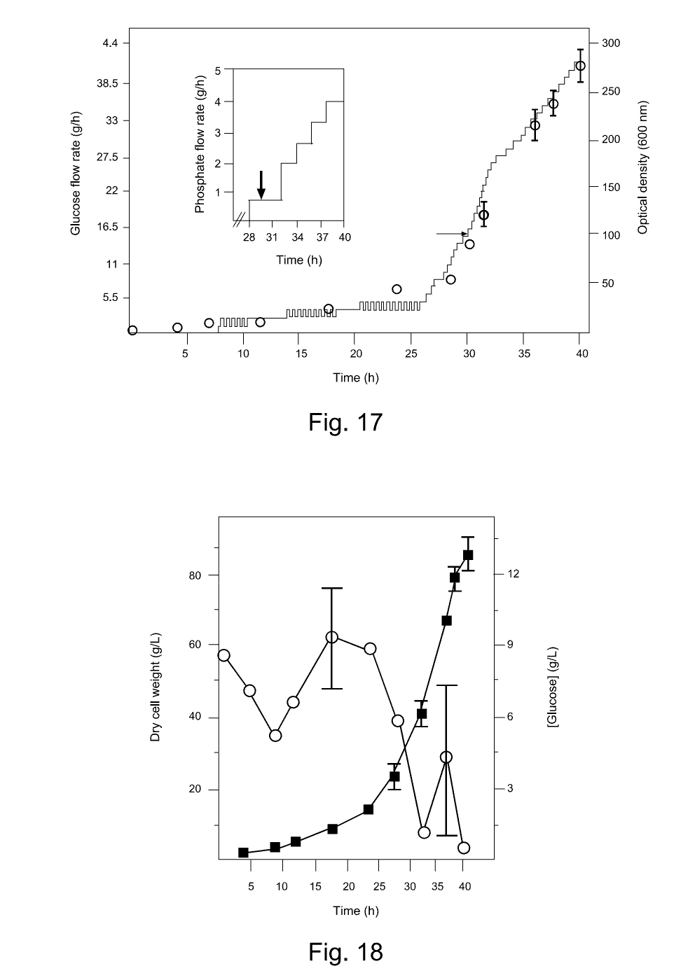

Referring now to FIG. 17, FIG. 17 shows a growth curve of BL21(DE3)/pTHBHug and flow rate of glucose and phosphate during bioreactor runs. Solid lines on the large and small graphs indicate flow rates of glucose and phosphate, respectively during a typical run. Circles indicate optical density of the culture at 600 nm. The optical density results are an average of three experiments and standard deviations below 6 OD are not shown. The arrow indicates the time of induction of the cultures with IPTG and the starting point of the heme/base feed.

Referring now to FIG. 18, FIG. 18 shows the glucose concentration (unfilled circles) and dry cell weight (filled squares) of cultures of BL21(DE3)/pTHBHug. The results are an average of three experiments and standard deviations below 1.5 g/L are not shown.

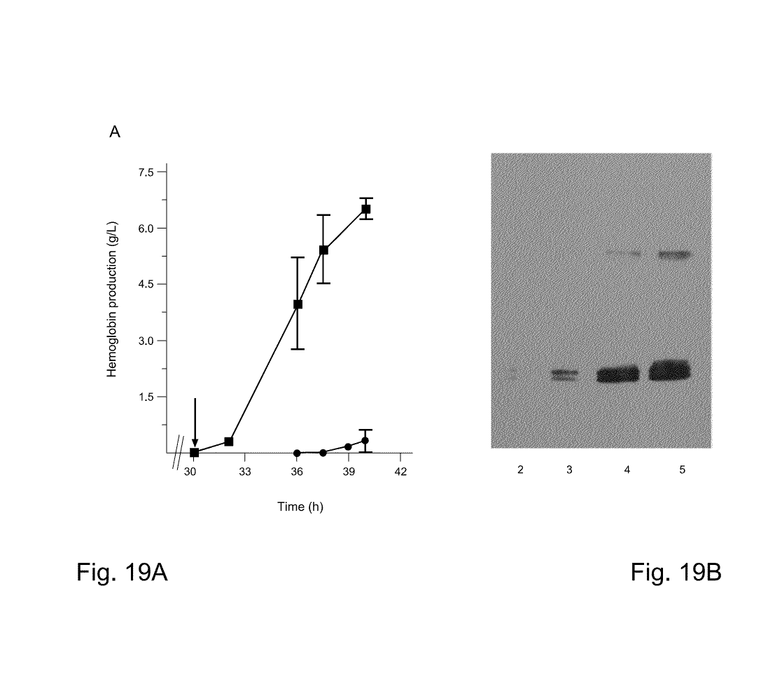

Referring now to FIG. 19, FIG. 19 shows the hemoglobin production of BL21(DE3)/pTHBHug. A. Graph of hemoglobin produced during bioreactor runs. Squares indicate concentrations of soluble hemoglobin isolated from cells and circles indicate concentration of hemoglobin released from cells that lysed during the run. The results are an average of three experiments, and standard deviations below 0.5 g/L are not shown. The arrow indicates time of induction. B. Immunoblot of hemoglobin produced from a representative run. Lane 1, hemoglobin control, 0.285 μg; lanes 2-5 contain samples taken at following time points: lane 2, 30 h (induction); lane 3, 32 h; lane 4, 37.5 h, and lane 5, 40 h. The equivalent of 0.014 μL of culture was loaded.

In one embodiment, the present disclosure provides a method for producing hemoglobin in a bioreactor comprising: providing a bioreactor containing a strain of E. coli comprising hemoglobin producing genes and heme transport genes on a one plasmid system; introducing a carbon source into the bioreactor; growing a culture of the E. coli strain; inducing the hemoglobin producing genes; and allowing the E. coli to produce hemoglobin.

The hemoglobin producing genes are induced when the culture reaches an OD600 of 80. In certain embodiments, the culture may be grown on a defined media or a complex media. The carbon source may comprise a glucose feed or a glycerol feed. In certain embodiments, the carbon source may comprise a glucose feed and a yeast extract. The carbon source may be introduced into the bioreactor at a rate calculated by using a mass flow equation. The method may further comprise introducing oxygen, base, and acid feeds into the bioreactor. The method may further comprise introducing a phosphate feed into the bioreactor. In certain embodiments, the phosphate feed may replace the acid feed. In certain embodiments, the phosphate feed may be introduced into the bioreactor once the culture reaches an OD600 of 90. In certain embodiments, the method may further comprise introducing a heme feed into the bioreactor. The amount of heme introduced into the bioreactor may be more than 0.21 mg/mL of culture. The method may further comprise introducing a metal feed into the bioreactor.

In another embodiment, the present disclosure provides a method for producing hemoglobin in a bioreactor comprising: providing a bioreactor containing a strain of E. coli comprising hemoglobin producing genes and heme transport genes on a one plasmid system; introducing oxygen, base, acid, and glucose feeds into the bioreactor; introducing a phosphate feed into the bioreactor; growing a culture of the E. coli strain; inducing the hemoglobin producing genes; and allowing the E. coli to produce hemoglobin.

The hemoglobin producing genes are induced when the culture reaches an OD600 of 80. The glucose feed may be introduced into the bioreactor at a rate calculated by using a mass flow equation. The phosphate feed may be introduced into the bioreactor once the culture reaches an OD600 of 90. The method may further comprise introducing a heme feed into the bioreactor. The method may further comprise introducing a metal feed into the bioreactor.

In another embodiment, the present disclosure provides a method for producing hemoglobin in a bioreactor comprising: providing a bioreactor containing a strain of E. coli comprising hemoglobin producing genes and heme transport genes on a one plasmid system; introducing a glucose feed into the bioreactor; introducing a heme feed comprising ammonium hydroxide into the bioreactor; growing a culture of the E. coli strain; inducing the hemoglobin producing genes; and allowing the E. coli to produce hemoglobin. In certain embodiments, the glucose feed may be introduced at a flow rate calculated by using a mass flow equation. In certain embodiments, the amount of heme introduced into the bioreactor is at least 0.21 mg/mL of the culture. In certain embodiments, the amount ammonium hydroxide introduced into the bioreactor is sufficient to maintain a pH of about 6.8 in the bioreactor. In certain embodiments, the hemoglobin producing genes are induced when the culture reaches an OD600 of 80.

To facilitate a better understanding of the present invention, the following examples of certain aspects of some embodiments are given. In no way should the following examples be read to limit, or define, the entire scope of the invention.

### EXAMPLES

The following Examples provide details in the development of the methods of producing human hemoglobin described herein.

### Example 1

One strategy for producing large quantities of a recombinant protein is to use a bioreactor for high cell density cultivation. High cell density cultivation may help to reduce the cost of producing recombinant proteins by increasing the volumetric yield (g L⁻¹) and reducing the amount of time required to produce large amounts of recombinant protein in a small volume which is called volumetric productivity (g L⁻¹ h⁻¹). E. coli is an ideal organism for recombinant protein production in bioreactors because of its well-characterized system and its high volumetric productivity of recombinant protein.

FIG. 1 illustrates an embodiment of a bioreactor in accordance with one embodiment of the present disclosure. Bioreactors may be used to maintain cell growth under optimal conditions for a long period of time. In certain embodiments, a bioreactor may use a pH probe, a dissolved oxygen probe, a temperature probe, and/or a foam probe to monitor the conditions inside the growth vessel. These probes may send data from the growth vessel to a computer controller which may then maintains the various conditions at their set values. The pH may be controlled by activation of a pump attached to an acid or base feed bottle. The dissolved oxygen may be controlled by adjusting the rotation rate of the impellers or changing the air to pure oxygen. Foam, which may be result of cell lysis, may be suppressed by activation of a pump attached to an anti-foam feed bottle. Temperature may be controlled by adjusting the temperature of a heating blanket that surrounds the vessel or by allowing cool water to flow through a small metal tube located inside of the vessel.

Nutrient feeding may be controlled by a time-based pump control program which runs on an attached computer. Many factors affect how well a high cell density culture grows and the amount of recombinant protein produced, such as oxygen availability, heat dissipation, nutrient selection and availability, feeding technique, acetic acid accumulation, and plasmid retention.

Oxygen Availability:

One gram of oxygen may be required for every 1.06 grams of E. coli. Growth and recombinant protein production may decrease as oxygen availability decreases. Using pure oxygen, agitating the culture, pressurizing the vessel, reducing the temperature, and limiting the amount of nutrients are techniques often used to help ensure the oxygen demands are met. The impellers used in the bioreactor may reduce the size of the oxygen bubbles which may increase their surface area to volume ratio allowing more gas to diffuse into the media. Pressurization of the vessel may increase the solubility of the oxygen. Reducing the growth temperature of a culture also may help to reduce oxygen demands by decreasing the growth rate and increasing the oxygen solubility. Decreasing the growth rate by nutrient limitation also may decrease the oxygen demand of a culture.

Heat Dissipation:

When bacteria reach high cell densities, they often produce large amounts of heat. Various temperature control methods may be used to dissipate this heat. In one method a cooling jacket may be placed around the vessel or a metal tube is present inside the vessel through which cold water flows. Sometimes the heat production is so great that the cooling mechanisms cannot keep pace; in this case, a reduction in the growth rate of the bacteria may help reduce the amount of heat produced.

Nutrient Selection and Availability:

The nutrients which are used and the amount of those nutrients which are available may determine the potential growth and recombinant protein production. Excess of the following nutrients may inhibit growth (average inhibiting concentration is shown in parentheses): glucose (50 g/L), ammonia (3 g/L), iron (1.15 g/L), magnesium (8.7 g/L), phosphorous (10 g/L), and zinc (0.038 g/L). To avoid growth limiting concentrations of nutrients, the initial media may contain the amount of nutrients required to begin bacterial growth and essential nutrients may be fed to the bacteria based on varying demand throughout the run. The feed solution may typically be simple and may consist of a carbon and/or a nitrogen source along with other nutrients involved in DNA synthesis and energy metabolism. Feed solutions with more concentrated carbon energy sources may yield higher final biomass by limiting the dilution of the culture by excess water addition. Defined media are typically used by most researchers because they usually provide more consistent growth and quantities of protein production.

However, some researchers have found that the use of undefined nutrients such as yeast extract may be required for optimal recombinant protein production. When glucose is used in conjunction with yeast extract, glucose may be used as an energy source and the yeast extract may provide the necessary precursors for cellular replication and protein production. Yeast extract may also help buffer the effects of varying bacterial growth rates by providing a second energy source should the glucose concentration run too low.

Feeding Technique:

The feeding technique used during a bioreactor run may largely determine the yield of biomass and recombinant protein. The types of feeding strategies include batch and fed-batch. Batch feeding may entail adding all of the nutrients required for an entire run to the starting media. Batch feeding may cause growth inhibition because the concentration of nutrients required for an entire run may be too high. However, the initial portion of a bioreactor run is considered a batch growth cycle because the bacteria are allowed to grow at their maximum rate until the initial carbon source is consumed. After the initial carbon source is consumed, a fed-batch (restricted growth) feeding strategy may be used. Fed-batch feeding adds the carbon and/or nitrogen source to the culture throughout the run in order to maintain a sub-inhibiting concentration of the required nutrients. The term fed-batch feeding encompasses many different approaches to glucose addition, including constant-rate feeding, stepwise feeding, exponential feeding, DO-stat, and pH-stat. Constant-rate feeding maintains the same rate of nutrient addition throughout the run but does not take into account the increasing nutrient demand as the concentration of bacteria increases. This may result in a continuously decreasing growth rate and thus a greatly reduced biomass and recombinant protein yield. Stepwise feeding may remedy the problems associated with constant-rate feeding by increasing the amount of nutrients added to the culture as the cell concentration increases. Gradually increasing the amount of glucose may allow the bacteria to grow at an exponential rate resulting in higher biomass and recombinant protein yields. However, determining what duration each step should be may be difficult often resulting in excess glucose accumulation. Excess glucose accumulation may lead to acetic acid production by E. coli which can inhibit growth and recombinant protein synthesis. It has been found that acetic acid production can be reduced by maintaining the bacteria at a slow constant growth rate. Exponential fed-batch feeding may allow for the control the growth rate of a culture by using a mass flow equation developed to more accurately predict the nutrient requirements of a given bacterial strain.

DO-stat and pH-stat were developed in order to indirectly measure the amount of glucose present in the media and respond to the changing conditions of the culture. DO-stat and pH-stat take advantage of the fact that a dramatic increase in dissolved oxygen or pH may be associated with depletion of the growth substrate. Once the dissolved oxygen or pH exceeds a certain value, a predetermined amount of nutrient may be added to the culture. Although the DO-stat method has provided a more precise method of nutrient addition, it should not be used in conjunction with anti-foam. Anti-foam may produce a rapid increase in dissolved oxygen when added to the media which will falsely trigger the addition of glucose.

Acetic Acid:

Acetic acid is one of the main inhibitory byproducts of E. coli growth and recombinant protein production in bioreactors. Any excess carbon energy source may be converted to acetic acid when the amount of the carbon energy source greatly exceeds the processing ability of the bacterium. Research has shown that saturation of the tricarboxylic acid cycle and/or the electron transport chain is the most likely cause of the acetic acid accumulation. Media choice may affect the level of acetic acid inhibition. Cells grown in defined media may be affected more than those grown in complex media. Replacement of glucose with glycerol may also greatly decrease the amount of acetic acid produced. It is believed that glycerol produces less acetic acid than glucose because its rate of transport into a cell is much slower than that of glucose. However, glycerol is more expensive than glucose, and may cause the bacteria to grow more slowly. The use of reduced growth temperatures can also decrease the speed of carbon source uptake and growth rate thus decreasing the production of acetic acid. E. coli produce acetic acid not only in the presence of an excess carbon energy source or during fast growth but also under anaerobic conditions. When E. coli are allowed to grow too fast, they may exceed the oxygen delivery ability of the bioreactor system which may lead to anaerobic growth conditions. To prevent this from happening, a slower constant growth rate may be maintained through nutrient limitation. Other methods for reducing acetic acid accumulation include genetic modification to prevent acetic acid production, addition of acetic acid utilization genes, and selection of strains with reduced acetic acid. BL21(DE3) is one of the strains that has been shown to produce lower levels of acetic acid because of its ability to use acetic acid in its glyoxylate shunt pathway.

Plasmid Retention:

Maintenance of plasmid copy number throughout the growth of a culture may be essential for high levels of recombinant protein production. Plasmid loss may be a problem with bioreactor cultures because they are grown for a much longer period of time than cultures grown in a shaker incubator. One of the problems caused by the length of a bioreactor run is that a majority of the antibiotics commonly used have a short half-life. To solve this problem, the antibiotic must be added at certain points throughout the run. Also, the length of the run may cause certain nutrients to be depleted which in turn results in plasmid loss. Depletion of magnesium, glucose, or phosphate may cause plasmid instability and loss. Solutions to nutrient depletion include decreasing the temperature of cultivation to reduce the metabolic rate, increasing the rate of nutrient addition, and adding the depleted nutrient through the feed line. Another method for increasing the plasmid copy number uses a runaway-replication plasmid that increases from one to one thousand copies if the temperature is increased from 30° C. to 42° C. after induction.

### Example 2

As previously mentioned, a mass flow equation was used to more accurately predict the nutrient requirements of a given bacterium. Below is the mass flow equation:

$${M}_{s} ( t ) = ( \frac{{μ}_{set}}{{Y}_{X / S}} + m ) * X ( {t}_{0} ) * V ( {t}_{0} ) * {e}^{{μ}_{set} * ( t - {t}_{0} )}$$

Mₛ(t) is the flow rate in grams/hour of glucose (or other carbon energy source) as a function of time that is required to maintain the bacteria at the specified growth rate.

μₛₑₜ is the specific growth rate (hr⁻¹) of the exponential (logarithmic) phase of cellular growth, the increase in cell mass per unit of time. This value may be set and it determines how fast the culture will grow. A value that has been shown to produce very little acetic acid is often used. For E. coli, is often set to 0.14 or 0.17. μ can be calculated by the following equation:

$$μ = \frac{ln ( 2 )}{{t}_{d}}$$

ln(2) is the natural logarithm of 2 and t_{d} is the doubling time of the cells. μ decreases as the doubling time increases.)

Y_{X/S} is the dry cell weight (DCW) yield on the carbon substrate (grams of DCW/grams of carbon). Y_{x/s} is a constant that has already been determined in prior papers. For E. coli growth on glucose it is 0.5 (g DCW/g carbon)

m is the specific maintenance coefficient. This is another constant that has been determined in earlier papers. The unit for is (g carbon/g DCW*hr). This value sets how much glucose is required to maintain the amount of dry cell weight the bioreactor has already accumulated for one hour. m is set to 0.025 (g carbon/g DCW*hr) for E. coli growth on glucose.

The X in X(t₀) stands for cell concentration (g DCW/L) and the (t₀) indicates the value used is the one from the start of the run. The reading that determines the value is typically an optical density reading at 600 nm (OD600). As described in the materials and methods, to convert this value into a (g DCW/L) value, a dry cell weight conversion value has to be determined for the spectrophotometer.

V(t₀) is the volume in liters at the start of the run.

e^{μset*(t-t0)} is the portion of the equation that takes into account the exponential nature of the growth of bacterial cultures. e is a mathematical constant and approximately equals 2.71828. This constant is raised to the power of μₛₑₜ*(t−t₀).

μₛₑₜ is the specific growth rate used earlier in the equation, and (t−t₀) calculates the amount of time that has passed since the start of the bioreactor run. t is set to the current time and t₀ is the time of the start of the run. The equation is set up so that the only changing variable is t, hence the term Mₛ(t) which is mass flow as a function of time.

Using the Results from the Mass Flow Equation:

After obtaining the values from the mass flow equation, these values must be converted from (g/hr) to a flow rate value for the carbon source pump on the bioreactor.

To obtain this value, a flow rate standard curve for glucose in mL/minute must be made. The mass flow values which are in (g/hr) must be divided by the concentration of glucose (or other carbon source) in grams per mL to obtain an mL per hour value. This mL per hour value must then be divided by 60 minutes per hour to covert to mL per minute. This value is used in the equation of the best fit line from the flow rate standard curve to obtain the flow rate settings used on the bioreactor program:

$$Flow rate = ( slope of flow rate standard curve ) * \frac{( \frac{{M}_{s} ( t ) ( \frac{g}{hr} )}{glucose concentration ( \frac{g}{hr} )} )}{60 ( \frac{min}{hr} )}$$

A flow rate standard curve must be constructed for use in converting the values of the mass flow equation to flow rate values. The flow rate value determines how much time the pump head is turning during a ten second period. A flow rate of ten turns the pump on for one second during a ten second cycle. Discussed below is one method of constructing a flow rate standard curve.

A flow rate standard curve may be constructed by first inserting the tubing that will be used for the nutrient feed bottle into the pump head. Then the tubing has to be primed (completely filled) with water. After the tube is primed, the pump is turned off. A timer and a 10 ml graduated cylinder are used to gather the data for the curve. Different flow rate settings are input into the bioreactor control tower, each setting being higher than the previous one. At least ten different flow rates ranging between 0.1 and 100 should be used to collect data for the curve. The pump is turned and the timer is started for each flow rate setting. The water is caught in the 10 mL graduated cylinder. After 5-10 mL has been collected, the pump is turned off and the timer is stopped. A weighing boat should be used to collect the water for flow rate settings between 0.1 and 1.0. This volume can then be measured with a 200 μL micropipette. This data is then graphed (see FIG. 2) with the first point being at the X Y origin (0,0). The equation of the best fit linear line for the data points is then used to convert the mass flow rate equation values to pump flow rate values.

### Example 3

### Bioreactor Culture Media

Trace Metal Mix 200 mL:

A trace metal mix may provide E. coli with essential nutrients required for growth. A 200 mL preparation may comprise the following: 0.26 g (9.538 mM) ZnCl₂, 0.4 g (8.406 mM) CoCl₂-6H₂O, 0.4 g (8.266 mM) Na₂MoO₄-2H₂O, 0.2 g (6.802 mM) CaCl₂-2H₂O, 0.254 g (5.255 mM) CuSO₄-5H₂O, 0.1 g (8.086 mM) boric acid (H₃BO₃), 20 mL of 12.1 M HCl (1.21 M final concentration). The HCl may be added to 150 mL of ultra-pure water and then all of the trace metals may be added. The solution may be filled to the 200 mL mark with ultra-pure water and may then be filter sterilized.

Iron Mix 200 mL:

The heme transport system genes of P. shigelloides are iron regulated meaning they are turned on more efficiently under low iron conditions. For this reason, FeCl₃ was separated from the trace metal mix to allow control over iron concentration if needed. The iron was routinely reduced in small scale hemoglobin assays in 5 mL of fermentation media, but was not reduced in cultures grown in the bioreactor so that there was not a limiting nutrient which could stop the growth of the culture. The heme transport genes may be expressed well when they are present on the same plasmid as the hemoglobin genes, and thus iron starvation may not be required for them to be well expressed. 200 mL of the iron solution contains 4.86 g (149.812 mM) of FeCl₃ and 20 mL of 12.1 M HCl (1.21 M final concentration). The HCl may be added to 150 mL of ultra-pure water and then the FeCl₃ may be added. The solution may be filled to the 200 mL mark with ultra-pure water and may then be filter sterilized.

Inoculum Media 1 L:

Inoculum media may be used in growing up the bacteria for inoculation of the bioreactor. One liter of inoculum media may comprise the following ingredients: 4.1 g (30.127 mM) KH₂PO₄ (monobasic), 7.0 g (40.189 mM) K₂HPO₄ (dibasic), 2.0 g (15.135 mM) (NH₄)₂SO₄, 1.0 g (3.400 mM) Trisodium citrate dihydrate (Sodium citrate tribasic dihydrate), 0.154 g (0.625 mM) MgSO₄-7H₂O, and 0.230 g (1.998 mM) proline. The above ingredients may be dissolved in 900 ml of ultra-pure water, and phosphoric acid may be used to pH this solution to 6.8. The solution may then be raised to 949 mL with ultra-pure water and autoclaved for 20 minutes on slow exhaust. After the solution has been autoclaved and cooled to room temperature, the following ingredients may be added: 20 mL of 10% (10 g/L) yeast extract (1.0% final concentration), 25 mL of 40% (400 g/L) glucose (1.0% final concentration), 3 mL of trace metal mix, 3 mL of iron mix, 312 μL of 0.285 g/mL thiamine (88.92 μg/mL final concentration), and 333.33 μL of 12.5 mg/mL tetracycline (4.166 μg/mL final concentration).

Fermentation Media with Yeast Extract 850 mL:

850 mL of fermentation media may be used for each run of the bioreactor and may contain the following ingredients: 1.7 g (14.696 mM) KH₂PO₄ (monobasic), 3.06 g (20.669 mM) K₂HPO₄ (dibasic), 1.7 g (15.135 mM) (NH₄)₂SO₄, 1.0 g (5.100 mM) Trisodium citrate dihydrate (Sodium citrate tribasic dihydrate), 0.1309 g (0.625 mM) MgSO₄-7H₂O, and 0.1955 g (1.998 mM) proline. The above ingredients may be dissolved in 800 ml of ultra-pure water, and phosphoric acid may be used to pH this solution to 6.8. The solution may then be raised to 807 mL with ultra-pure water and autoclaved for 20 minutes on slow exhaust. After the solution has been autoclaved and cooled to room temperature, the following ingredients may be added: 17 mL of 10% (10 g/L) yeast extract (1.0% final concentration), 21.25 mL of 40% (400 g/L) glucose (1.0% final concentration), 2.55 mL of trace metal mix, 2.55 mL of iron mix, 265.2 μL of 0.285 g/mL thiamine (88.92 μg/mL final concentration), and 142 μL of 12.5 mg/mL tetracycline (2.083 μg/mL final concentration).

Bioreactor Feed Bottle Solutions

5.0 M Phosphoric Acid:

Phosphoric acid may be used to decrease the pH of the culture if it exceeds 6.9. The stock concentration of phosphoric acid may be 14.7 M. Multiplying the desired volume of 5.0 M phosphoric acid by (5/14.7) yields the volume of acid required. Subtracting this volume from the desired volume of 5.0 M phosphoric acid yields the volume of ultra-pure water required to dilute the concentrated phosphoric acid.

5.0 M Ammonium Hydroxide:

Ammonium hydroxide may be used to increase the pH of the culture if it decreases below 6.7. The stock concentration of ammonium hydroxide is 14.8 M. Multiplying the desired volume of 5.0 M ammonium hydroxide by (5/14.8) yields the volume of base required. Subtracting this volume from the desired volume of 5.0 M ammonium hydroxide yields the volume of ultra-pure water required to dilute the concentrated ammonium hydroxide.

Anti-Foam 1:1.5:

The length of bioreactor runs and low nutrient concentrations may cause cell lysis to occur. When cells lyse, foaming occurs which if left unchecked can clog the gas exhaust port causing contamination issues and potential over pressurization of the vessel. Anti-foam may be used to decrease the amount of foam being produced. To ensure proper mixing on a stir plate, Sigma anti-foam 204 may be diluted with water in a ratio of 1:1.5. This formula for the antifoam mixture was used initially, then later changed to 1:2.5 anti-foam water mix because the higher concentration of antifoam inhibited the growth of the culture. The antifoam may be autoclaved once and the tubing for the antifoam may be changed every three runs to prevent toxic reactions that slow down the growth of the strain.

Glucose 79.5% with 0.485% Yeast Extract:

The glucose and yeast extract mixture may provide the necessary nutrients for continued bacterial growth and DNA replication. 82.5% glucose solution, which is subsequently diluted to 79.5% glucose by additives, may be made in 1 liter quantities in order to provide enough glucose for a bioreactor run. The glucose solution may be prepped in a feed bottle to ensure no volume loss as a result of transferring the solution to another container. 250 mL of ultra-pure water may be brought to a rolling boil so that the glucose dissolves easily; 82.5% glucose is the maximum solubility of glucose at room temperature. Then ten 82.5 gram portions of glucose may be added to the boiling water. While the glucose is being added, the solution may be continuously stirred and heated. After all of the glucose has been added, 5.0 grams of yeast extract may be added to the solution so that the concentration of yeast extract is 0.5%. After the yeast extract has dissolved, ultra-pure water may be added to the solution until it is at the 1 liter mark. This solution may then be autoclaved for 10 minutes on slow exhaust. After the solution is autoclaved and cooled to room temperature, 542.5 μL of 0.285 g/mL (0.845 M) thiamine, which is heat sensitive, may be added to the solution. Then 30.9275 mL of 2/3 g/ml (2.705 M) MgSO₄-7H₂O may be added to the solution. The composition of the final glucose solution may be: 795 g/L glucose, 4.85 g/L yeast extract, 20 g/L (81.144 mM) MgSO₄-7H₂O, and 0.15 g/L (0.445 mM) thiamine. To prevent the glucose solution from crystallizing in the lines going into the bioreactor vessel, the glucose may be heated and stirred on a hot plate as it is being fed into the bioreactor vessel.

Thiamine Stock Solution (0.285 g/mL or 0.845 M) 10 mL:

Thiamine may be a necessary nutrient in most bioreactor glucose feeds. 10 mL of thiamine may be made at a time. To make the 0.845 M thiamine stock solution, 2.85 grams of thiamine hydrochloride may be added to 6 mL of ultra-pure water in a sterile 15 mL graduated container and vortexed on high. Then ultra-pure water may be added till the solution is around the 9 mL mark. The solution may be vortexed on high until all of the thiamine goes into solution. Then the thiamine solution may be filled up to the 10 mL mark with ultra-pure water. A 10 mL syringe with a filter sterilizing tip may then be used to filter the thiamine solution into another sterile 15 mL tube.

MgSO₄-7H₂O Stock Solution (0.667 g/mL or 2.705 M) 250 mL:

The magnesium sulfate heptahydrate solution may be used to provide magnesium for DNA replication. 200 mL of boiling ultra-pure water may be added to a 250 mL bottle with 166.67 grams of MgSO₄-7H₂O in it. This may then be shaken and allowed to stir for 5 to 10 minutes. Then boiling ultra-pure water may be added till the solution is at the 250 mL mark. Boiling water may ensure that the high concentration of MgSO4-7H₂O goes into solution easily. After the MgSO4-7H₂O has fully dissolved, the solution may be autoclaved for 20 minutes on slow exhaust.

Phosphate Solution 1 L:

When bacteria grow for a long time in the bioreactor, they may use up the phosphate that was present in the media at the beginning of the run. When this occurs, the bacteria may be unable to use the glucose present in the media which can lead to a spike in the glucose concentration and the eventual death of the culture. To prevent this from happening, a phosphate feed may be required. The phosphate feed may comprise: 327 g/L (2.403 M) KH₂PO₄ (monobasic) and 139.809 g/L (0.803 M) K₂HPO₄ (dibasic). The first step to making the phosphate solution may be boiling 400 mL of ultra-pure water so that KH₂PO₄ (monobasic), the least soluble ingredient, will easily go into solution. In addition to boiling the water, KH₂PO₄ (monobasic) may be added before K₂HPO₄ (dibasic) because of its lower solubility. Then the K₂HPO₄ (dibasic) may be added to the solution. Room temperature ultra-pure water may then be added till the solution is around the 900 mL mark. 14.8 M ammonium hydroxide may be used to pH the solution to 6.8 (the same pH as the fermentation media). The solution may then be filled to the 1 L mark.

Bioreactor Operation Procedures

Autoclaving the Bioreactor:

Prior to autoclaving the bioreactor, the pH probe may be calibrated with 4.0 and 7.0 pH buffers. Then a protective cap may be placed on top of the pH probe to prevent steam from damaging the exposed metal contact. After the pH probe is calibrated, the dissolved oxygen probe membrane may need to be cleaned and have new O₂ electrolyte added to it. The dissolved oxygen probe and impeller shaft also may have protective caps which may need to be placed on them to prevent steam damage. A sample collection tube that has a glass vial attached to it may be located on top of the bioreactor. This glass vial may need to be turned until it is slightly loose or it will potentially crack during autoclaving. Also, there may be a metal exhaust condenser located on top of the bioreactor. To maintain sterility of the vessel, an air filter may be attached to the end of the condenser. The temperature probe may be removed from the bioreactor and does not have to be autoclaved because it may be placed in a metal well and does not contact the inside of the vessel. The foam probe should be pushed down till only ½ an inch of it is above the top of the bioreactor. This position allows the foam probe to be tested after media is added to the vessel. All air filters may be wrapped in foil to prevent the steam from damaging them. The ends of all of the addition and extraction tubes may be placed in plastic centrifuge tubes and all of the silicon tubes, except the glucose tube, may be clamped to ensure sterility of the vessel after autoclaving. Before the bioreactor is autoclaved, about 100 mL of ultra-pure water may be added to the vessel to ensure steam sterilization of the interior of the vessel. The vessel may then be autoclaved for 45 minutes on slow exhaust. After the autoclaving cycle finishes, the glucose feed tube (which may be left open to prevent over pressurization of the vessel) may be immediately clamped shut to prevent contamination.

Setup after Autoclaving:

Several steps may be required to setup the bioreactor after it is autoclaved. These steps may be performed the day before inoculation of the bioreactor. First, the following probes may need to be reattached to the bioreactor control tower: pH probe, dissolved oxygen probe, and foam probe. The temperature probe may also need to be placed into the thermal well. After the probes are reattached, the thermal blanket and the water cooling tubes may be reattached. To ensure an adequate supply of oxygen for the bacteria, the following steps may performed: reattaching the air tube, turning on the ambient air pump, opening the valve to the pure oxygen tank, and placing the motor on the impeller shaft. Next, the filter on the condenser may be removed and a foam trap jar may be attached to it. The foam trap jar may help to prevent over pressurization of the vessel by catching any foam that passes the exhaust condenser. After everything is reattached, the following external control devices may be turned on: the water cooling pump and the bioreactor control tower. The next step may be to drain the 100 mL of ultra-pure water from the vessel through the harvest tube and to add fermentation media to the vessel using an autoclaved funnel. Following the media addition, the bioreactor may be set up for maximum air saturation so the dissolved oxygen probe can be calibrated the next day. The following settings for maximum air saturation may be input into the bioreactor control tower: an agitation rate of 800 rpm, a max gas flow rate of 2.2 liters per minute, and a temperature of 36.5° C.

Growing and Preparing the Inoculum:

An L-agar plate with 12.5 μg/mL tetracycline may be streaked with BL21(DE3)/pHb0.0TM1hug, which contains a plasmid (small circular extrachromosomal DNA) with the P. shigelloides heme transport system genes and a triple mutant version of the human hemoglobin genes which encodes more stable hemoglobin. This plate may be incubated at 37° C. for 20 hours. The plate may be refrigerated for several hours and then 10 isolated medium sized colonies may be scraped off the plate with a sterile toothpick. If only one colony is used, the culture may not grow consistently in the bioreactor, because the colony may be a slow grower or a faster than normal grower. Inconsistent growth that occurs when a single colony is used may make it difficult to control glucose levels in the vessel where the culture is grown. The toothpick may then be scraped inside of a microfuge tube that has 1 mL of sterile saline in it. After the colonies are scraped into the microfuge tube, the tube may be vortexed on the highest agitation setting in order to suspend the bacteria in the saline. Then, a 1:9 dilution of the culture may be made in sterile saline, and 40 μL, 20 μL, 5 μL, and 1 μL aliquots from the microfuge tube may be used to inoculate four separate Erlenmeyer flasks that contain 160 mL of inoculum media with 4.166 μg/mL tetracycline. The Erlenmeyer flasks may then be incubated at 37° C. for 18 hours in a shaker incubator at 225 rpm. The OD600 of the cultures may be measured at 18 hours. If one of them has an OD600 of 2.50 it may be used for inoculating the bioreactor. The OD of the culture was determined on a 1:9 dilution in saline of a portion of the culture. Otherwise, the cultures may be incubated for another 30 minutes to 1 hour depending on the measured ODs. When the flask is chosen, an OD600 of a 1/10th dilution of the culture may be taken (100 μL of culture in 900 μL of saline). This diluted OD600 may be taken because the spectrophotometer is more accurate when the readings are between 0.2 and 0.45. Next, the culture may be spun down in order to reduce the volume of liquid added to the bioreactor. The chosen culture may be split into five centrifuge tubes each containing 32 mL of culture and is spun down in a centrifuge for 7 minutes at 5500 rpm. After the culture is spun down, the supernatant may be poured off and each of the pellets may be resuspended in 1 mL of inoculation media with 4.166 μg/mL tetracycline. The reduced volume inoculum may then be used for inoculating the bioreactor.

Inoculation Setup:

Before bacteria can be grown in the vessel, the dissolved oxygen probe and pH probe may need to be calibrated. The temperature should be at 36.5° C. and the dissolved oxygen percent should be steady. To calibrate the dissolved oxygen probe, the value that the probe reads may be input into the dissolved oxygen span value (maximum value) of the bioreactor control tower as 100% dissolved oxygen. Once the span value is set, the dissolved oxygen probe may be disconnected until the raw output reading stabilizes and that value may be set to 0% in the set zero option. To recalibrate the pH probe, a sample may be taken from the vessel and an external pH probe may be used to measure the pH of the sample. This reading may then be input into the bioreactor as the zero value which represents the current pH of the media. After calibrating the pH probe, the foam probe may be tested by switching pump A to foam detection. If the foam probe is properly connected, it should activate pump A. The foam probe may then be raised even with the black gasket on the top lip of the bioreactor so that it will detect foam before it enters the exhaust condenser. The anti-foam feed bottle may then be placed on a stir plate to keep the solution homogeneous throughout the run. Once the anti-foam mixture is homogeneous, the anti-foam tube may be placed into pump A which is turned on manual at 100%. This may allow the anti-foam tube to be primed (completely filled) so that anti-foam will be immediately added to the bioreactor if the foam probe is triggered. When priming the anti-foam tube, the anti-foam may be permitted to reach the tube of smaller diameter on the triport and then be turned off. If the anti-foam is permitted to go further, it may enter the vessel and alter the initial growth of the culture. After the anti-foam tube is primed, pump A may be set to foam detection with an 80% sensitivity setting and a setpoint of 0.5 (the pump flow rate in this case). The sensitivity setting may keep the pump from activating if the foam probe is splashed by the media but no foam is present.

Other tubes that require priming include: acid, base, and glucose. Priming of the acid and base feed can be accomplished by changing the pH multiplier (pump flow rate) to 100 and changing the pH setpoint so that the acid or base pump will be activated. When the acid or base reaches the end of their tubes, the associated pump may be turned off. At this point, the values for pH control may be entered. A setpoint, the value that determines the pH that will be maintained, may be entered into the pH control. In this case, the setpoint value may be 6.8. The multiplier may be set to 0.75 to reduce the amount of acid or base added to the culture. The last value that may be set is known as the deadband; this value determines how much the pH can vary from the setpoint before the acid or base pump is activated. In order to bring the pH of the solution up to 6.8, the deadband may need to be set to zero and the acid and base pumps may need to be turned on. Once the pH of the media hits 6.8, the deadband value may be changed to 0.1 so that the acid pump will activate at 6.9 and the base pump will activate at 6.7. Allowing the pH to vary between 6.7 and 6.9 may help reduce the amount of acid and base added to the culture.

Next, control parameters for the dissolved oxygen response system may need to be set up. The Bioflo 110 bioreactor from New Brunswick Scientific uses a cascade dissolved oxygen response system. The cascade system allows the user to determine the order in which different methods of raising the dissolved oxygen level will be used. For example, the Agit/02/Air option will first raise the agitation in response to the dissolved oxygen decreasing below the set value. If the agitation rate hits the set maximum and it is not sufficient to maintain the dissolved oxygen level, the air pump will start mixing in pure oxygen with the ambient air until it is at 100% pure oxygen. If the dissolved oxygen is still too low, the gas flow rate will be increased until it hits the maximum gas flow limit. The following values may be set for the cascade program:

Cascade: Agit/02/Air

Agit Hi limit: 1200 rpm

Agit Lo limit: 250 rpm

Gas Hi Limit: 2.2

Gas Lo Limit: 2.2

The dissolved oxygen cascade control program may take control over the agitation and gas flow programs when they are set to auto so no values are set for those programs. In addition to setting the cascade values, the dissolved oxygen setpoint (the dissolved oxygen percent that is maintained) may be set to 40% of maximum air saturation. At induction, the Gas Hi Limit value may be changed to 3.2 to allow more gas exchange and the foam probe sensitivity may be changed from 80% to 60% and the foam probe lowered to 2 marks above the 1 L mark on the vessel.

The final step in setting the bioreactor control tower up may be to set pump B to manual control and a setpoint (pump flow rate) of zero. This may allow an attached laptop to control the flow rate of pump B which is used for pumping the glucose feed. On the laptop there may be bioreactor control program called Biocommand Plus. Before the start of each run, a preprogrammed batch “recipe” may be loaded and a file name with the batch number on it is designated for storage of probe and run data. Batch recipes contain programs which have “time profiles” that can be set to change the flow rate of a pump at specific times within a run. The time profile for the pump B program may be based on the results of the mass flow equation for the strain of E. coli. After the data file is designated, the run may be started with the Biocommand Plus program. Around five hours into the run, the glucose solution may be placed on a hot plate and it is set to 50° C. with a stir rate of 2 out of 9. If the glucose solution is not heated and stirred, it may crystallize in the tube and cause no glucose to enter the bioreactor. After 1 hour, the glucose feed tube may be placed into pump B and hooked into the glucose feed port. The tube may then be primed by changing the flow rate of the 0 to 7 hour section of the time profile to 100 and turning the pump off once the glucose reaches the bioreactor. To prevent bubbles from forming in the metal tube that attaches the silicon glucose feed tube to the vessel, the glucose feed control may be switched off and the metal tube unscrewed when the glucose reaches this tube. The part of the metal tube that screws into the vessel may then be turned up side down and the glucose feed control switched back on until glucose bubbles out of the metal tube. Then the metal tube may be screwed back into the vessel, and the pump head turned until drops of glucose may be seen entering the vessel. After the glucose feed tube is primed, the flow rate value of the 0 to 7 hour section may be changed back to 0 and pump B may be turned on again.

Mid-Run Procedure:

Around 25 hours into the run, the acid feed pump may be changed to a phosphate solution feed pump. At this point in the run the acid feed may no longer be required. The phosphate feed on the other hand may be needed when the culture reaches an OD600 of 90. Around an OD600 of 115, the original amount of phosphate in the media is likely to have been consumed. Without phosphate, the bacteria will be unable to use the glucose to continue to grow. The pump switching step may be required because the Bioflo 110 only has 4 pump heads so the acid pump has to be repurposed for use as a phosphate solution pump. The tubing for the acid feed pump may be emptied and then the tubing for the phosphate solution feed may be inserted into the acid feed pump. After the tubing is switched, the cable for control of the acid pump may be moved to pump C control on the tower. Then pump C may be set to manual control. The time profile program on the laptop may control pump C. This program may be started along with the batch at the beginning of the run and may be set to activate pump C around the time the culture reaches an OD600 of 90. The flow rates used in the program may be based off the particular phosphate feed. After pump C is set to manual control, the tubing may be primed by changing the flow rate of the 0 hour section of the time profile to 100 and the pump may be turned off once the phosphate solution reaches the bioreactor. After the phosphate solution feed tube is primed, the flow rate value of the 0 hour section may be changed back to 0 and pump C is turned on again.

Also at around 25 hours into the run (sooner if needed), the multiplier value for the pH control may be changed to 2.5. This increases the flow rate of the base pump so it can keep up with the increased pH demands as a result of accumulation of bacteria.

Hemoglobin Induction:

Induction of the hemoglobin genes (turning on their transcription) may occur when the culture reaches an OD600 of approximately 80. Before the hemoglobin genes are induced, the base feed tubing may be emptied and may be primed with a heme solution that contains 0.562 g of heme dissolved in 375 mL of 5.0 M ammonium hydroxide (1.5 mg/mL heme). When heme is added to the media, the P. shigelloides heme transport system is used by BL21(DE3)/pHb0.0TM1hug to transport the heme into the cell which is then incorporated into the human hemoglobin proteins. As was the case with the phosphate solution feed, the base feed pump may need to be repurposed because of the limited number of pump heads so that it both adds heme and controls the pH. Using the base feed as a heme/base feed has the added benefit of adding heme at a faster rate as the concentration of cells increase in the bioreactor. As the concentration of cells increases, more metabolic byproducts may be produced so more base may be added to the media to maintain the pH within acceptable levels. Priming of the base feed may require the pH deadband to be set to 0 so that the pH will increase to 6.75. This may help prevent the pH from decreasing below 6.7 while the base solution is being changed to the heme solution. After the base tubing is emptied, the tubing may be placed into the heme solution bottle and the pH multiplier is set to 100 in order to prime the tubing. When the heme solution reaches the bioreactor, the pH multiplier may be changed back to 2.5 and the deadband may be set to 0.1. Also, the heme tube and bottle may be wrapped in foil and a nylon mesh because heme is light sensitive.

Once the heme solution tube is primed, an IPTG and tetracycline mix may be injected with a sterile needle and syringe through the rubber septum port on top of the bioreactor. The IPTG and tetracycline mix may contain 2 mL of 200 mg/mL IPTG (final concentration around 0.333 mg/mL) and 150 μL of 12.5 mg/mL tetracycline (final concentration around 2.083 μg/mL). IPTG may serve as a gratuitous inducer that turns on the expression of the human hemoglobin genes, which may be under the control of a tac promoter. The tetracycline may need to be added because the culture continuously increases in volume which dilutes the amount of tetracycline originally added to the media. The amount added to the culture helps to maintain the tetracycline concentration around 2.083 μg/mL. This helps to reduce plasmid loss during the run.

Cleaning the Bioreactor:

After the bioreactor run finishes, cleaning may be required to prepare it for the next run. One method of cleaning the bioreactor is described below.

First, all of the feed lines are emptied into their feed bottles by removing them from their pump heads and clamping all of the tubes (including the exhaust tube) except for the tube being emptied. This allows pressure to build in the vessel which forces the solutions back into the feed bottles. Once all of the feed lines are cleared, they (along with the foam trap tube) are taken and hooked into to a distilled water tap in order to completely rinse them out. The last step in cleaning the tubing requires ethanol to be added to the anti-foam tubing. Anti-foam is not fully soluble in water so ethanol must be used to dissolve any of the anti-foam residue remaining in the tubing. This ethanol is then rinsed out using the distilled water tap. After all of the tubes and bottles are cleaned, the attached air filters and ends of the tubes are wrapped in foil. These are then autoclaved on slow exhaust for 20 minutes. The silicon antifoam tubing may be changed every three runs, otherwise a residue from the tubing may become toxic to the cells. The glucose feed tubing may be changed every run, otherwise the glucose flow rate may be inconsistent.

Next, the bioreactor vessel is cleaned. First the culture is drained through the harvest tube into a 5 liter bottle so that it can be autoclaved and then four washing steps are performed. If the draining of the culture vessel may be performed late at night, then the vessel may be filled with 3 L of distilled water and allowed to sit overnight to prevent the heme in the culture from damaging the membrane in the oxygen probe. During each of the washing steps, the sample collection tube is flushed four times into a glass vial using the attached syringe. After the culture is drained, the vessel is filled with 3 liters of distilled water and the agitation is turned to 1200 rpm. This step is 30 minutes long and it helps remove any loose bacterial debris present in the vessel. The water from this step is sterilized by the addition of bleach. After 30 minutes, the vessel is drained and 3 liters of 1 M NaOH is added to it. The NaOH removes any baked on bacterial debris and heme. This step lasts for one hour and the agitation is again set to 1200 rpm. After 1 hour, the NaOH is drained and two ten minute washings with 3 liters of distilled water each are performed in order to wash out any residual NaOH. Once the last distilled water washing is performed, the water is left in the vessel until it is autoclaved, unless the vessel sits for several days before its next use. Then it may be drained and left empty so that the oxygen probe membrane is not damaged. The last step is to remove the pH probe and place its tip into a cap with 4.0 pH buffer in it. The dissolved oxygen probe is left in the vessel to maintain the moistness of its membrane.

Plasmid and Glucose Concentration Analysis

Plasmid Isolation and Glucose Concentration Analysis:

Maintenance of plasmid copy number throughout the growth of a bioreactor culture may be essential in order to produce large quantities of hemoglobin. High plasmid copy number may result in high production levels of heme transport proteins. This in turn may result in high quantities of heme being transported into the cell which is then incorporated into the large amounts of hemoglobin being produced. If the plasmid copy number is too low, the heme transport proteins and human hemoglobin proteins may be produced at low levels. The reduced amount of heme transport proteins may result in reduced amounts of heme entering the cell. Reduced heme concentration in the cell reduces the amount of properly formed hemoglobin produced.

Samples may need to be taken at induction and at the end of the run to ensure the plasmid count is not decreasing. After the samples are taken, a plasmid DNA isolation may need to be performed so the plasmid concentration can be determined. The sample collection tube located on top of the bioreactor may be used to withdraw a sample from the bioreactor vessel. Before a sample is taken, the sample tube may need to be flushed in order to ensure only fresh cells are collected. The tube may be cleared of dead cells that accumulated from the previous sample by using the attached syringe to withdraw approximately 1 mL of culture through the tube into a waste collection vial. Before taking the sample, the culture should be drawn into the sample tube and pushed back into the vessel three or four times to ensure the sample is entirely fresh. After the tube has been cleared, a sterile collection vial may be used to take a 5 mL sample from the culture. 1 mL of this sample may be centrifuged in a microcentrifuge for 5 minutes at 13,200 rpm and an Accu-Check® Compact Plus glucose monitor may be used to analyze the mg/dL glucose concentration of 50 μL of the supernatant. A decrease in plasmid concentration is usually a result of glucose starvation at some point during the run. After the glucose concentration is determined, the OD₆₀₀ of the sample may be determined from a series of dilutions of 100 μL of the sample; the rest of the sample may remain on ice so the cells will not lyse. The following dilutions may be used: 1/10th (100 μL of culture and 900 μL of saline), 1/100th (100 μL of the 1/10th dilution and 900 μL of saline), 1/200th (50 μL of the 1/10th dilution and 950 μL of saline), 1/500th (200 μL of the 1/100th dilution and 800 μL of saline), and 1/1000th (100 μL of the 1/100th dilution and 900 μL of saline). The OD600 of the dilution that falls within the 0.2 to 0.45 range (where the spectrophotometer is most accurate) may be recorded. Then 6.0 OD units worth of cells may be centrifuged for 5 minutes at 13,200 rpm. The supernatant may then be removed and a QIAGEN® Spin Miniprep Kit may be used to purify and isolate the plasmid DNA.

The bacterial pellet may be resuspended with 250 μL of 4° C. Buffer P1 that contains RNase A. Then 250 μL of alkaline lysis Buffer P2 may be added to the microfuge tube containing the resuspended pellet and the tube may be inverted 10 times. After this step, 350 μL of neutralization/high-salt Buffer N3 may be added to the tube, inverted 10 times, and it may be centrifuged for 10 minutes at 13200 rpm in order to remove chromosomal and cellular debris from the sample. Proteins, chromosomal DNA, and cellular debris may end up in the pellet and the plasmid DNA may end up in the supernatant. Approximately 850 μL of supernatant is removed from the tube and transferred to a QIAprep silica-gel membrane column. This column may then centrifuged for 1 minute at 13,200 rpm in order to isolate the plasmid DNA in the silica-gel membrane. The flow-through may be discarded and 500 μL of Buffer PB may be added to the column to remove any remaining endonucleases. This may be centrifuged for 1 minute at 13,200 rpm and the flow-through may be discarded again. Then 750 μL of ethanol-containing Buffer PE may be added to the column which may be subsequently centrifuged for 1 minute at 13,200 rpm to remove salts. The flow-through may be discarded and the column may be centrifuged for 1 more minute in order to remove residual Buffer PE. In the last step, the column may be moved into a new microfuge tube and 100 μL of 70° C. Buffer EB may be added directly on top of the silica-gel membrane (heating the EB increases plasmid recovery). The column may then be allowed to set for 1 minute and may then be centrifuged for 1 minute at 13,200 rpm to elute the plasmid DNA into the microfuge tube.

Plasmid Concentration Analysis:

Plasmid DNA isolations are usually performed on the inoculum, on the sample prior to induction, and on the sample at the end of the run. 3 μL of the plasmid DNA from these samples may be diluted by 1/10th with 27 μL of 1×TE buffer (1.0 mM Na₂EDTA and 10 mM Tris) and then 3 μL of this dilution may be analyzed using agarose gel electrophoresis. A photograph of the gel may be taken with an AutoChemi bioimaging system using Labworks 4.0 software. This photograph may be analyzed to determine if the plasmid concentration of the bioreactor samples remained at the plasmid concentration level found in the inoculum sample. If the plasmid concentration is high the plasmid count is also high. To ensure maximum hemoglobin production, there should be very little decrease in plasmid concentration across all of the samples.

Dry Cell Weight Determination

In the mass flow equation, the OD600 reading must be converted into a (g DCW/L) value so that it can be used in the mass flow equation. The first step in converting the OD600 value into a (g DCW/L) value may be to determine the dry cell weight conversion value for the spectrophotometer. The dry cell weight conversion value may be determined by obtaining multiple OD600 readings. In addition to obtaining multiple OD600 readings, 3-5 mL of each OD600 sample may be pipetted into a microfuge tube and spun down in a centrifuge for five minutes at 13,200 rpm. The microfuge tubes used for spinning down samples may be dried at 80° C. for 24 hours and weighed prior to sample addition. Drying the centrifuge tubes removes any excess moisture in the tubes and allows the weight of the samples to be accurately determined after they are dried. After the samples are spun down, the supernatant may be removed. Then they may be placed in a microfuge tube rack with their tops open and dried for 24 hours at 80° C. Once 24 hours have passed, the dried samples may be weighed along with their tubes. The data acquired from the samples may then be input into the following equation to determine the dry cell weight conversion value:

$$DCW conversation value ( \frac{g \frac{DCW}{L}}{OD 600} ) = \frac{( \frac{( ( Tube + dry cells ) ( g ) ) - Tube ( g )}{mL of sample centrifuged} ) * 1000 ( \frac{mL}{L} )}{OD 600}$$

The (Tube+dry cells) (g) value is the weight of the tube which the cells were dried in plus the weight of the cells inside the tube. Subtracting the weight of the tube, which was dried and weighed prior to adding and drying the cells, from the weight of the tube and cells together yields the dry cell weight. Then the dry cell weight value is divided by the mL of culture that were spun down before drying the cells in order to convert the dry cell weight to (g DCW/ml). Multiplying this value by 1000 (mL/L) converts the value to (g DCW/L). Dividing this value by the OD600 reading gives the dry cell weight conversion value. The average of multiple dry cell weight conversion values is then used to obtain the dry cell weight conversion value for the spectrophotometer. The dry cell weight conversion value is then used to convert the OD600 values to dry cell weight values:

$$X ( {t}_{0} ) = cell concentration ( \frac{gDCW}{L} ) = DCW conversation value ( \frac{g / L}{OD 600} ) * OD 600 reading$$

Hemoglobin Soluble Protein Assay

The amount of soluble hemoglobin present in a bioreactor sample may be calculated by using a spectrophotometer to determine the 419 nm absorbance value (OD419) of the soluble protein fraction of the sample. The 419 nm wavelength corresponds to the maximum absorbance peak of carboxyhemoglobin (HbCO). Prior to determining the OD419, the sample may be fully saturated with carbon monoxide. This may ensure that the degree of absorbance of HbCO is directly proportional to the concentration of hemoglobin produced by the bacteria. Carbon monoxide is used because it has a strong affinity for hemoglobin and causes it to produce a stronger absorbance than hemoglobin alone. This allows for finer resolution between samples of similar hemoglobin concentration.

Bioreactor samples are taken and OD600 values may be determined in the same manner as described in the plasmid isolation procedure. A sample is usually taken at the start of induction to determine if there is any hemoglobin production prior to induction and at the end of the run to determine the amount of hemoglobin produced during induction. Once the OD600 value is determined, 5 OD units worth of the sample may be spun down at 4° C. for 5 minutes at 13,200 rpm in the refrigerated centrifuge. The amount of the sample required to equal 5 OD units may be determined by dividing 5 by the OD600 value. Next, a 125 mL bottle of 0.1M pH 7.5 Tris may be bubbled with carbon monoxide for 20 minutes on ice. Then the pellets, which are resting on ice, may be resuspended with 1 mL of carbon monoxide bubbled Tris by vortexing them on the highest setting. The resuspended pellets may then be placed on ice for 10 minutes. After 10 minutes have passed, the resuspended pellets may be spun down at 4° C. for 5 minutes at 13,200 rpm. The supernatant may be removed and the pellets may be placed into the −80° C. freezer and frozen overnight.

The pellets may then be thawed on ice and 450 μL of 0.1 M pH 8.0. Tris, which may be placed on ice and have carbon monoxide bubbled through it for 20 minutes, may be added to each tube. Then the pellets may be resuspended by vortexing them on the highest setting. The resuspended pellets may then be placed on ice for 10 minutes. After 10 minutes have passed, 75 μL of Lysozyme (final concentration, 1.36 mg/mL) may be added to each tube in order to lyse the bacterial cells and release the hemoglobin they contain. In addition to Lysozyme, 5 μL of the protease inhibitor phenylmethanesulfonylfluoride (PMSF) (final concentration, 0.9 mM) may be added to each tube to prevent any bacterial proteases from degrading the hemoglobin. After PMSF and Lysozyme are added to each tube, they may be vortexed on the highest setting for 20 seconds. The tubes may then be placed on ice for 30 minutes. Next, the tubes may be placed on their side on top of ice and rotated for 10 minutes at a speed setting of 5 on a VWR OS-500 orbital shaker. In order to further clarify the supernatant, 4.5 μL of DNAse (final concentration, 9 μg/mL) may be added to digest any bacterial DNA that is present and 20 μL of 1/4 Triton X100 (final concentration, 0.9%) may be added to emulsify the bacterial membranes. This solution may then be vortexed on the highest setting for 20 seconds. After the solution is vortexed, the tubes may again be placed on their side on top of ice and rotated for 10 minutes at a speed setting of 5 on the orbital shaker. Following the 10 minute rotation, the microfuge tubes may be spun down at 4° C. for 15 minutes at 13,200 rpm. The supernatants may then be pipetted into new microfuge tubes.

In order to analyze the amount of soluble hemoglobin, the sample may be diluted to allow an OD419 reading of between 0.3 and 1.0, the range determined to allow the most reliable and repeatable data. Also, a new cuvette may be used to determine the OD419 in order to ensure the most accurate reading possible. The OD₄₁₉ may then be used in conjunction with the equation for the Beer-Lambert law to determine the g/L concentration of hemoglobin in the bioreactor samples. The Beer-Lambert law states that the light absorbance (A) of a material is based on the following variables: the molar extinction coefficient of the material (ε) (how well the material absorbs light at a certain wavelength), the concentration of the material (C) (usually in molar or millimolar units), and the distance the light travels through the material (d) (path length). The value used for the molar extinction coefficient of carboxyhemoglobin is 191 mM⁻¹×cm⁻¹. In order to determine the concentration (C) of hemoglobin in the solution, the Beer-Lambert equation A=εCd may be rearranged to: C=A/εd.

The path length may be set to 1 cm for the spectrophotometer. The absorbance value may be adjusted so that it reflects the dilution of the supernatant and the volume of cells spun down to equal 5 OD600 units:

A=((OD₄₁₉ of diluted supernatant×dilution factor)−(OD₄₁₉ of background))×(OD₆₀₀ of sample/5 OD₆₀₀ units)

The OD419 absorbance value of the diluted supernatant is multiplied by the dilution factor to reflection any dilution of the supernatant with Tris. Then the average undiluted OD419 value for the background strain BL21(DE3)/pHb0.0-hug is subtracted from this value to remove any absorbance not resulting from hemoglobin. BL21(DE3)/pHb0.0-hug is the BL21(DE3) strain of E. coli which has been transformed with the pHb0.0-hug plasmid. This plasmid was constructed by deleting the human hemoglobin genes from the pHb0.0 plasmid and inserting the P. shigelloides heme transport system genes. After the background has been subtracted, the adjusted OD419 value is multiplied by the OD600 of the sample divided by 5 OD600 units. This step accounts for the volume of cells that were spun down to equal 5 OD600 units; it converts the absorbance value for the supernatant to what the absorbance value would be if 1 mL of the sample was spun down for the supernatant assay. After the absorbance value is determined, it is divided by εd (191) to determine the concentration of hemoglobin in millimolar (mM) units. In order to determine the concentration of hemoglobin in g/L, the mM (millimole per liter) concentration value must be multiplied by 64.5 grams of hemoglobin per millimole (the millimolecular weight of hemoglobin).

### Example 4

The following example describes how certain experiments performed in accordance with certain embodiments of the present disclosure.

A human hemoglobin expression system that used the E. coli strain BL21(DE3) which had been transformed with the pHb0.0TM1hug plasmid has previously been developed. The pHb0.0TM1hug plasmid (See FIG. 3) has a pUC origin of replication and contains a tetracycline resistance gene, the P. shigelloides heme transport system genes (which encode HugABCD, TonB, and ExbBD), and a triple mutant version of the human hemoglobin genes (TM1). The triple mutant version of human hemoglobin (TM1) has one alpha (a) globin mutation (glycine at position 15 was changed to alanine) and two beta ((3) globin mutations (glycine at position 16 was changed to alanine and histidine at position 116 was changed to isoleucine) which make it more stable than wild-type human hemoglobin.

In a small scale hemoglobin production method a shaker incubator was used to grow a 5 mL culture of BL21(DE3)/pHb0.0TM1hug in a test tube for 8 hours at 36.5° C. at a rotation of 225 rpm. After 8 hours heme and IPTG were added to the culture and allow it to grow for an additional 16 hours. The heme was transported into the E. coli cells by the P. shigelloides heme transport system where it enhances production of recombinant triple mutant human hemoglobin. IPTG acted as a gratuitous inducer that turns on the expression of the triple mutant human hemoglobin genes, which were under the control of a tac promoter (a hybrid of trp and lac promoters).

A goal of this example was to determine how to move from a small scale (5 mL) human hemoglobin production method conducted on cultures grown in a test tube to a large scale (multiple liters) human hemoglobin production method conducted on cultures grown in a bioreactor. With the bioreactor large quantities of protein may not be produced unless the cell density level of the culture is very high. Before high cell densities are obtainable, a nutrient feeding strategy was developed. A nutrient feeding strategy was used to control how fast the bacteria grow and was designed to meet the nutrient requirements of the bacteria at different stages of growth. The rate of nutrient addition to the culture determined the rate of growth of the culture. A nutrient addition rate that is too high can lead to accumulation of growth inhibitory levels of glucose (around 2% or higher in many strains of E. coli). On the other hand, a nutrient addition rate that is too low can lead to glucose starvation and subsequent death of the culture. In determining the nutrient addition strategy, it was also taken into consideration which strain was being used and what plasmid the strain contained as both of these factors affect growth in the bioreactor. The following sections describe attempts at various nutrient feeding strategies and subsequent understanding concerning the growth and nutrient demands of the E. coli strain used that led to the development of a final nutrient feed and hemoglobin production strategy. Thus, the strategy involved two major components: working out the growth conditions required to achieve maximum cell densities and working on a strategy to get the cells to produce large quantities of hemoglobin.

Batch/Constant-Rate Feeding

All of the work that is described in this example was performed with the E. coli strain BL21(DE3) containing human hemoglobin genes and heme transport system genes on the plasmid pHb0.0TM1hug. In the initial attempts at working out growth conditions, the E. coli strain was grown with a mixture of batch feeding (a large amount of glucose is added in a short period of time at set intervals) and constant-rate feeding (continuous addition of glucose at a set rate). The feeding schedule shown in FIG. 4 was based on early attempts at determining the glucose feed schedule needed for hemoglobin producing E. coli. For the first 14.5 hours the feed was a batch style feed. The feed was programmed to add 6 mL of 40% glucose (2.4 g) every hour starting at 1.5 hours with the last addition being at the 14.5 hour mark. At 15 hours the glucose feed was programmed to switch to a constant flow rate of 1.0 (2.358 mL/minute or 0.0943 g/minute). The constant-rate portion of the feeding strategy was intended for use after the cells were induced to make hemoglobin; it was assumed the bacterial growth rate would decrease as a result of the bacteria converting a majority of the available glucose to energy for use in hemoglobin production. It was also assumed that a continuous flow of glucose would be required to ensure steady rates of hemoglobin production. The glucose feed was stopped at 24 hours because cultures are grown for 24 hours with the test tube hemoglobin production method.

The first attempts at working out growth conditions using this approach resulted in the culture achieving a maximum OD600 of only 5. There were two flaws with this glucose addition scheme. The first flaw was the use of batch addition of glucose at the beginning of the run. In the batch addition technique large quantities of glucose are added in a short amount of time at set intervals. Between the glucose additions, the bacteria reduce the glucose concentration in the culture to a point where the next addition does not lead to accumulation of excess glucose. However, the drastic swings in glucose concentration associated with the batch addition technique disrupted the normal growth of the bacteria resulting in an unhealthy culture. Constant-rate feeding is the other flaw in this glucose addition scheme. With constant-rate feeding, the nutrient addition rate does not take into account the increasing nutrient demand resulting from increasing concentrations of bacteria. This resulted in a continuously decreasing growth rate and eventually cell death.

In order to solve the problems associated with the batch/constant-rate addition technique, a stepwise addition technique was chosen for the next attempt at finding an appropriate nutrient feeding strategy. In the stepwise addition technique the glucose was added continuously in order to prevent drastic swings in glucose concentration. In addition to a continuous flow of glucose into the culture, the glucose flow rate of the stepwise addition technique increases as the cellular concentration increases. At this point it was determined that a glucose monitor would be useful to monitor the glucose levels in the culture.

Stepwise Glucose Feeding

After the culture reached a maximum OD600 of only 5 with batch/constant-rate feeding, the glucose feeding technique was changed to a slightly modified version of a stepwise glucose addition method described, as shown in FIG. 5. The goal was to grow a culture that reached an OD600 of 30. This disclosure discusses an early attempt at using E. coli to produce human hemoglobin in a bioreactor. An OD600 of 30.2 was reached by using the stepwise glucose feed; the OD600 of the culture was subsequently increased to 53 by concentrating the inoculum so that an inoculum culture of 112.5 mL in volume was centrifuged, resuspended in 5 mL of media, and used to inoculate the fermentation media in the bioreactor. A concentrated inoculum reduced the volume added to the bioreactor which helped in increasing the final OD600 of the culture.

As previously described, stepwise feeding, unlike constant-rate feeding, increases the amount of glucose added to the culture as the cell concentration increases. The glucose feed protocol shown in FIG. 5 was designed to provide increasing amounts of glucose until 5 hours. After 5 hours, the glucose flow rate was decreased based on the assumption that the cells would have reached their maximum possible OD600 and would require only maintenance levels of glucose. However, it was found that the stepwise feed described in previous references were was designed for an E. coli strain that grew much faster than BL21(DE3)/pHb0.0TM1hug. The slower growth rate of BL21(DE3)/pHb0.0TM1hug caused glucose to accumulate to high concentrations in the media. As shown in Table 1, the concentration of glucose in the culture reached 1.72% (17.2 g/L) at 3 hours and remained around 2% (20 g/L) or more until 26.25 hours.

TABLE 1

| Time (hrs) | OD600 | Glucose (g/L) |
|---|---|---|
| 0 | 0.243 | 10 |
| 1 | 0.28 | 10.8 |
| 2 | 0.383 | 11.2 |
| 3 | 0.489 | 17.2 |
| 4 | 0.657 | 28.2 |
| 5 | 0.752 | 37.2 |
| 6 | 0.885 | 43.2 |
| 7 | 1.42 | 47.7 |
| 11.5 | 3.59 | 49.4 |
| 22.75 | 30.4 | 28.1 |
| 24.25 | 40.7 | 18.6 |
| 26.25 | 46.3 | 12.3 |
| 28.75 | 53 | 2.5 |
| 30.5 | 52.4 | 0.1 |

Glucose concentrations around 2% or higher can inhibit growth of our E. coli strain. Even though the goal of reaching an OD600 of 30 was surpassed by concentrating the inoculum and using a stepwise feeding strategy, the culture was unhealthy and died soon after the maximum OD600 of 53 was reached. The low glucose flow rate at the end of the run when the bacteria were entering early exponential growth resulted in glucose starvation and the subsequent death of the culture. That the cells were in exponential growth was indicated by the fact that between 26.25 and 28.75 hours, the glucose concentration decreased by almost 1% (9.8 g/L) (Table 1) which indicates the cells were growing rapidly and requiring more energy. To provide sufficient amounts of glucose during early exponential growth and prevent subsequent glucose starvation, the glucose flow rate should be rapidly increased to match the rapidly increasing glucose consumption rate. However, instead of increasing the glucose flow rate from 26 to 29 hours, glucose flow rate was decreased to the lowest possible level (FIG. 5). The glucose flow rate was significantly lower than the glucose consumption rate which resulted in cell death from glucose starvation.

This run demonstrated that the stepwise feeding technique was too imprecise for maintaining non-inhibitory levels of glucose and that the use of it would involve too much guesswork. It also showed that BL21(DE3)/pHb0.0TM1hug reaches exponential growth at a much later time than the DH10B E. coli strain used in prior references. It was decided to switch to a glucose feeding strategy based on the mass flow equation in order to better match the glucose feeding rate to the nutrient requirements of the bacteria.

Mass Flow Equation Based Glucose Feed

From the results of the stepwise glucose feed (Table 1) it was found that the concentration of glucose in the culture reached inhibitory levels due to the period of exponential growth for our strain of E. coli occurring much later than expected. In order to reduce the accumulation of glucose, the next glucose feed was based on the mass flow equation. The mass flow equation was used to calculate how fast the glucose flow rate needed to increase to match the rate of growth of our strain of E. coli. The goal was to grow a culture that reached an OD600 close to the maximum OD600 of 246 (128 g/L dry cell weight) which is equivalent to an OD600 of 297.67 on our spectrophotometer. The equivalent OD600 was based on a 0.43 dry cell weight conversion value for the spectrophotometer (the value used to convert from OD600 to dry cell weight) versus a 0.52 dry cell weight conversion value for their spectrophotometer.

Through the use of the first mass flow equation based glucose feed (FIG. 6 blue line) and a 40% glucose feed solution a culture was grown to a maximum OD600 of 93 (Table 2). Even though this result was a significant improvement over the maximum OD600 of 53 achieved with a stepwise glucose feed (Table 1), a maximum OD600 of 93 is low when compared to the OD600 of 297.67 previously reported. There were 3 potential reasons why the culture reached a maximum OD600 of only 93: (1) The feed solutions used for the bioreactor may have been too dilute. (2) The glucose flow rate was too high. (3) The inoculum culture size was too small.

Feed solutions that are too dilute can decrease the maximum potential OD600 of a culture by adding too much water volume. To solve this problem, the concentration of all of the feed solutions were increased. Anti-foam was increased to a concentration of 1 part anti-foam to 1.5 parts water from a previous concentration of 1 part anti-foam to 2 parts water. The ammonium hydroxide base feed concentration was increased from 3.5 M to 5 M. The glucose feed concentration was increased from 40% to 79.5%.

The high flow rate of the first mass flow equation based glucose feed contributed to the accumulation of glucose to growth inhibiting levels of 2% or higher from 26 to 53 hours (Table 2).

TABLE 2

| Time (hrs) | OD600 | Glucose (g/L) |
|---|---|---|
| 0 | 0.230 | 10.3 |
| 1 | 0.306 | 10.2 |
| 2 | 0.420 | 9.7 |
| 3 | 0.559 | 9.7 |
| 5 | 0.752 | 9.5 |
| 6 | 0.875 | 9.4 |
| 7 | 0.968 | 9.4 |
| 8.25 | 1.104 | 9.1 |
| 26 | 5.6 | 19.8 |
| 27 | 6.1 | 23.3 |
| 28 | 7.1 | 27.5 |
| 29.33 | 8.5 | 31.8 |
| 52 | 93 | No Reading |
| | | Taken |
| 53 | 82 | 17.3 |

To solve this problem, the rate at which glucose was added to the culture was decreased so that the concentration of glucose would remain at 0.05% (0.5 g/L) or lower (FIG. 7 yellow line). This decision was based on the previous results where it was shown that maintaining a glucose concentration around 0.05% reduced accumulation of acetic acid in the culture to a point where the growth of the culture was not inhibited. The red line in FIG. 7 represents the flow rate values of the first mass flow equation based feed shown in FIG. 6 that have been converted from 40% glucose feed values to 79.5% glucose feed values to facilitate a direct comparison between the first (FIG. 6 blue line) and second (FIG. 7 yellow line) mass flow equation based glucose feeds.

Another factor that contributed to the accumulation of growth inhibiting levels of glucose was the size of the first inoculum culture. The bacteria population was too small to use the amount of glucose that was added to the media. To solve this problem the volume of the inoculum culture was increased from 112.5 mL to 160 mL. The volume of the inoculum was concentrated as before to 5 mL. By increasing the size of the inoculum culture the number of bacteria using the glucose present in the culture was also increased which in turn should result in a decrease in the glucose concentration. As expected, increasing the initial size of the bacteria population resulted in a reduction of the glucose concentration to 6.8 g/L by 9 hours (Table 3) for the second mass flow equation culture where glucose was added starting at 7 hours (FIG. 7 yellow line).

TABLE 3

| Time (hrs) | OD600 | Glucose (g/L) |
|---|---|---|
| 0 | 0.369 | 10.8 |
| 9 | 2.6 | 6.8 |
| 33 | 30.8 | 3.2 |
| 46.75 | 79.6 | 1.6 |
| 48 | 92.1 | 3.7 |
| 50.25 | 110 | 3.6 |
| 51.25 | 114 | 1.2 |
| 52.25 | 116 | 0.7 |

This was considerably lower than the first mass flow equation culture which had a glucose concentration of 9.1 g/L at 8.25 hours (Table 2) even though glucose was not added to the culture until 10 hours into the run (FIG. 6 blue line and FIG. 7 red line).

After all of the changes made to the bioreactor growth strategy, only a maximum OD600 of only 116 was achieved (Table 3). However, the glucose accumulation in the media was drastically decreased to an average of 0.23% (2.3 g/L) (Table 3) during the later portion of the second mass flow equation based run (33 to 52.25 hours) as opposed to an average of 2.39% (23.9 g/L) (Table 2: 26 to 53 hours) for the first mass flow equation based run. Even though 0.23% was almost 5 times the optimal concentration of 0.05% previously reported, a large number of dead cells present at the bottom of pellets of late run samples were noticed. This suggested that the strain of E. coli (BL21(DE3)/pHb0.0TM1hug) was not able to tolerate the low glucose conditions that were optimal for the E. coli strain used previously. In addition to dead cells being present in the pellets of the bioreactor samples, the rate of increase of the OD600 values drastically slowed down between 50.25 and 52.25 hours (Table 3). The most likely cause for this reduced rate of cellular growth was the decrease in glucose flow rate that occurred during this time. The glucose flow rate was decreased from 49.25 to 52.25 hours (FIG. 7 yellow line) in anticipation of the culture requiring less glucose as a result of it being in the stationary growth phase (reduced growth rate phase). However, instead of the culture being in the stationary phase of the growth cycle it was still in the exponential growth phase. Evidence that demonstrates the cells were in the exponential growth phase prior to the decrease of the glucose flow rate is shown in Table 3 where the OD600 value increased by 17.9 between 48 and 50.25 hours. After the decrease in glucose flow rate the OD600 value increased by only 6 between 50.25 and 52.25 hours. In order to reduce the number of dead cells present in late run samples and increase the maximum OD600, the rate of glucose addition during the late portion of subsequent runs was increased. However, this resulted in cultures with final OD600 values lower than 116 (data not shown) and pellets which still contained dead cells. After failing to increase the maximum OD600 by increasing the rate of glucose addition, it was decided to add yeast extract to the media and glucose feed. The reasoning was that if the glucose concentration became too low the bacteria would have yeast extract to use as a second energy source which would increase the maximum OD600 by preventing the cell death that occurred during the later stages of the low glucose runs.

Yeast Extract, Phosphoric Acid, and Phosphate Feeds

It was decided to add yeast extract to the media and glucose feeds based on the previous results where it was shown that yeast extract reduced acetic acid accumulation and promoted utilization of acetic acid when the glucose concentration of the culture reached low levels. It was also previously demonstrated that the use of yeast extract increased recombinant protein yields and provided a second form of energy for the bacteria. At the same time yeast extract was added to the media and glucose feed, a 5.0 M phosphoric acid feed was also added to the bioreactor setup. The phosphoric acid feed was added so that the pH could be reduced if the bacteria switched to using yeast extract as their main energy source as a result of glucose starvation. Breakdown of yeast extract (which contains a nitrogenous energy source) resulted in the production of basic waste products which caused the pH of the culture to increase.

In the first run where yeast extract was used the starting media contained 1% (10 g/L) yeast extract and the glucose feed contained 16.005% (160.05 g/L) yeast extract. The feeding strategy used for this run was the same as the second mass flow equation based feed (FIG. 7 yellow line) except there was no decrease in the glucose flow rate at the end of the run. During this run the culture reached a maximum OD600 of 181 (data not shown). While an OD600 of 181 is much higher than an OD600 of 116 it is still considerably lower than the OD600 of 297.67 previously reported. Even though a 16.005% yeast extract concentration is considerably lower than the concentration of yeast extract previously used, a large number of lysed cells in the pellets of late run samples were noticed. In an attempt to reduce the number of dead cells and further increase the maximum OD600, the yeast extract concentration in the glucose feed was reduced. The concentration of yeast extract in the starting media was kept at 1% throughout all of the yeast extract runs. After reducing the glucose feed yeast extract concentration to 0.97% (9.7 g/L), a culture was grown to a maximum OD600 of 255. However, a second culture grown using 0.97% yeast extract in the glucose feed reached a maximum OD600 of only 100 (data not shown). Unsure of what caused the drastic decrease in the maximum OD600, the yeast extract concentration in the glucose feed was decreased it to 0.485% (4.85 g/L). Instead of the culture growing to an OD600 comparable to 255, the culture reached a maximum OD600 of only 137 (data not shown). A second attempt at replicating the results of the first 0.97% yeast extract run also failed; the culture grew to a maximum OD600 of only 128.7 (Table 4).

TABLE 4

| Time (hrs) | OD600 | Glucose (g/L) |
|---|---|---|
| 20.25 | 15.4 | Glucose not |
| | | detectable |
| 24.5 | 19.5 | Glucose not |
| | | detectable |
| 25.75 | 21 | Glucose not |
| | | detectable |
| 28.5 | 27.5 | 0.08 |
| 44.5 | 76.9 | 0.08 |
| 47.75 | 124.7 | 17.5 |
| 48.5 | 128.7 | 44.5 |

Further analysis of the data of the 16.005% yeast extract run where the culture grew to a maximum OD600 of 181 and the data of the first 0.97% yeast extract run where the culture grew to a maximum OD600 of 255 were conducted. In the pH graphs of both of the runs it was noticed that large quantities of phosphoric acid were added to the cultures. The pH graph for the first 0.97% yeast extract run (255 OD600 run) is shown in FIG. 8 with periods of phosphoric acid addition highlighted in red. Excessive quantities of phosphoric acid were added to the cultures because the pH control program was not set up properly for yeast extract use. However, the pH control program was set up properly for all of the runs that followed the first 0.97% yeast extract run (255 OD600 run). With the new pH control program, all subsequent cultures received very little phosphoric acid. This decrease in phosphoric acid addition coincided with the drastic decrease in maximum OD600 values. This indicated that the initial phosphate present in the media at the beginning of the run was completely depleted by the end of the run.

Phosphate depletion, which is likely to occur in long bioreactor runs, is a serious problem for E. coli which require phosphate for glucose uptake and metabolism. E. coli transport glucose through an inner membrane transport protein that adds a phosphate group to glucose converting it to glucose-6-phosphate. This step is required for transport of glucose into the cytoplasm. E. coli also cannot use glucose for energy production unless it is in the form of glucose-6-phosphate in the cytoplasm. Two events will occur if the E. coli in the bioreactor cultures are no longer able to uptake and metabolize glucose: (1) The glucose concentration will suddenly spike as a result of the constant addition of glucose to the culture. (2) The growth rate of the bacteria will suddenly decrease. Table 4 shows the glucose concentration spike that occurred 47.75 hours into the third 0.97% yeast extract run. At 47.75 hours the glucose concentration was 1.75% (17.5 g/L) when it had been at 0.008% (0.08 g/L) only 3 hours earlier. At 48.5 hours (only 45 minutes later) the glucose concentration increased to 4.45% (44.5 g/L). Table 4 also shows the decrease in growth rate that occurred during the third 0.97% yeast extract run. Prior to the decrease in growth rate there was a period of rapid growth between 44.5 and 47.75 hours when the OD600 of the culture increased by 47.8. However, between 47.75 and 48.5 hours the OD600 of the culture only increased by 4. The growth rate decreased from 14.7 OD600 units per hour to 5.3 OD600 units per hour. Without the ability to uptake glucose and use it as an energy source the culture rapidly died.

To solve the phosphate depletion problem a phosphate feed was added to the bioreactor setup based on the phosphate feed previously described. At the same time, the second mass flow equation based feed (FIGS. 7 and 9 yellow line) was modified so that the glucose flow rate increased instead of decreased at the end of the run (FIG. 9 purple line). With the phosphate feed and modified version of the second mass flow equation a culture was grown to a maximum OD600 of 277. After the first phosphate feed run, cultures were consistently grown to an OD600 around 265. Also, with the addition of the phosphate feed there was no sudden spike in the glucose concentration and no sudden decrease in the growth rate (Table 5).

TABLE 5

| Time (hrs) | OD600 | Glucose (g/L) |
|---|---|---|
| 23 | 25.3 | 1.34 |
| 29.5 | 37.1 | 1.01 |
| 45 | 91.4 | 0.05 |
| 47.5 | 122.4 | 0.05 |
| 50.07 | 172 | 0.17 |
| 52.82 | 216 | 0.2 |
| 58.25 | 277 | 0.27 |

Human Hemoglobin Production and Plasmid Loss

After the growth conditions required to achieve high cell densities were determined, a strategy was developed for production of large quantities of human hemoglobin. There were four questions that had to be answered to develop the hemoglobin production strategy: (1) At what point in the culture's growth is it best to induce the human hemoglobin genes? (2) How much IPTG is needed to fully induce the human hemoglobin genes of all of the bacteria in the culture? (3) How much heme is required to ensure maximum hemoglobin production? (4) How fast should heme be added to the culture?

During the early development of the large scale human hemoglobin production strategy, the focus was on finding the ideal phase of the culture's growth cycle for induction of the human hemoglobin genes. For the first hemoglobin production run, the human hemoglobin genes were induced during the late exponential growth phase of the culture (OD600 of 172). The late exponential growth phase was chosen because induction is described as a period where a large metabolic burden is placed on the cells which results in a drastically decreased growth rate. Also, it is recommended that induction occur once the culture is near the maximum OD600. After the human hemoglobin genes were induced, the culture grew to a maximum OD600 of 270 and was a light red color indicating hemoglobin production (data not shown). The growth rate of the culture was unaffected by induction of the human hemoglobin genes. Surprised by this result, it was decided to induce the human hemoglobin genes of the next culture during the early exponential growth phase (OD600 of 32.2). This culture grew to a maximum OD600 of 265 but the culture was brown indicating minimal hemoglobin production (data not shown). Deciding that induction was too early, the human hemoglobin genes of the next culture were induced during the mid-exponential growth phase (OD600 of 94.80). This culture grew to an OD600 of 253 and was a dark red color indicating large amounts of hemoglobin were produced (data not shown). It was decided that the mid-exponential growth phase was the optimum phase of the culture's growth cycle to induce the human hemoglobin genes.

To answer the question of how much IPTG should be added to the culture, a quantity of IPTG was used that was based on the optimum IPTG concentration for the test tube hemoglobin production method. When the test tube hemoglobin assays were performed with the strain of E. coli, it was found that cultures where the human hemoglobin genes were induced with an IPTG concentration of 5 μg/mL produced the largest amount of hemoglobin. To calculate how many grams of IPTG should be added to the bioreactor, the facts that test tube cultures typically grow to a maximum OD600 value between 3 and 4 and bioreactor cultures typically grow to a maximum OD600 value between 250 and 275 were taken into account. This means the cell density of bioreactor cultures is 60 to 90 times the cell density of test tube cultures. Based on the increased cell density and the final volume of the culture being around 1500 mL, it was calculated that 0.4 grams of IPTG would be required to fully induce the human hemoglobin genes of all the bacteria in the culture. The final concentration of IPTG was 0.267 mg/mL which was 53.4 times the concentration of IPTG used in the test tube. This concentration of IPTG was used for the runs where it was determined the best phase of growth for induction of the human hemoglobin genes. After ideal phase of growth for induction of the human hemoglobin genes was found, the quantity of IPTG that was added to the culture was increased to see if more hemoglobin would be produced. The human hemoglobin genes were initially induced with 0.4 grams of IPTG as before but then 0.2 grams of IPTG every 2 were added hours until 1.4 grams of IPTG had been added to the culture. The final concentration of IPTG was 0.933 mg/mL which was 186.6 times the concentration of IPTG used in the test tube. This high concentration of IPTG caused a rapid decrease in the growth rate (the culture grew to a maximum OD600 of only 145) and resulted in minimal hemoglobin production (the culture was brown). After discovering that it was possible to kill a culture with too much IPTG, the previously successful IPTG concentration of 0.267 mg/mL for induction of the human hemoglobin genes was returned to.

Prior to starting the development of the bioreactor hemoglobin production strategy, test tube hemoglobin assay was used to determine that the optimal heme concentration for this strain of E. coli was 3.75 μg/mL (It was later realized that 3.0 μg/mL of heme may be optimal as it created a lower level of background in the BL21(DE3) containing a plasmid with the heme transport genes but no hemoglobin genes.) This concentration was used as a starting point for calculating the quantity of heme required for maximum hemoglobin production in a bioreactor culture. The optimal heme concentration for a test tube culture was multiplied by 53.4 (the same factor used to increase the IPTG) to account for the high cell density of a bioreactor culture. The 1500 mL final volume of the bioreactor culture was then taken into account to arrive at 0.3 grams of heme being required for maximum hemoglobin production (final concentration of 0.2 mg/mL). Once the quantity of heme that should be added to the bioreactor was determined, the rate of adding it to the reactor had to be determined. Two factors that determined how fast heme could be added to the bioreactor were that it must be dissolved in ammonium hydroxide and the maximum solubility of heme in 3.0 M ammonium hydroxide is 18 mg/mL. To add 0.3 grams of heme to the bioreactor, 16.7 mL of 3.0 M ammonium hydroxide would also have to be added. If 16.7 mL of 3.0 M ammonium hydroxide were rapidly added to the bioreactor culture, the pH of the culture would drastically increase and a large number of cells would die. Assuming the culture could survive this, the quantity of heme per OD600 unit would be higher than wanted because the culture would be at an OD600 between 80 and 100 instead of the maximum OD600 of 250 to 275 on which the calculations were based.

It was determined that the best way to solve these problems was to dissolve 0.4 g of heme in 200 mL of 5.0 M ammonium hydroxide (2 mg/mL) and use this mixture for the base feed during induction. The concentration of heme used (2.0 mg/mL) was based on 150 mL of ammonium hydroxide being added to the culture during the last 14 hours of the run (the period of induction). By using the base feed as a heme feed, an excessive change in the pH of the culture and an excessive amount of heme being added to the culture were avoided. With a heme feed controlled by the pH program, the rate of heme addition was gradual and increased as the cellular density of the culture increased; higher cellular concentrations produce more metabolic byproducts which in turn require larger quantities of base to maintain the pH of the culture. A base feed with this concentration of heme (2.0 mg/mL) was used for the runs where the best time for induction of the human hemoglobin genes and the amount of IPTG to use were determined.

By inducing the human hemoglobin genes with 0.4 g of IPTG during mid-exponential growth phase and using a heme feed concentration of 2.0 mg/mL the reddest culture yet was grown (highest hemoglobin producing culture). However, an attempt to duplicate these results was made, a brown culture (minimal hemoglobin production) was obtained. This sudden lack of hemoglobin production was most likely a result of plasmid loss. As previously mentioned, plasmid loss will result in reduced expression levels of the heme transport system genes which will cause a decrease in the quantity of heme entering the cell. If there is a reduction in the quantity of heme entering the cell, the quantity of properly formed hemoglobin will also be reduced. In addition to a reduced amount of heme transport proteins, plasmid loss will cause a reduction in the expression level of the human hemoglobin genes which will further reduce hemoglobin production.

To prevent plasmid loss, the initial concentration of tetracycline in the starting media was increased. By increasing concentration of tetracycline the bacteria were forced to retain more copies of the plasmid (pHb0.0TM1hug) which contains a tetracycline resistance gene. The initial tetracycline concentration was increased from 2.08 μg/mL to 8.332 μg/mL and 12.498 μg/mL; both of these concentrations caused drastic decreases in the maximum OD600 of the cultures which ranged from 25 to 90 OD600 (data not shown). After these undesirable results, additional tetracycline was added at the time of induction instead of adding all of it to the starting media. The quantity of tetracycline added to the culture was based on how much the culture had been diluted from the addition of the feed solutions. By returning the culture to the beginning tetracycline concentration (2.08 μg/mL) there was better plasmid retention and the maximum OD600 values were unaffected (data not shown).

Believing that the plasmid loss problems were solved, the next step was the optimization of the concentration of heme in the base feed. Optimization of the heme concentration in the base feed began by decreasing the heme concentration. The heme concentration was decreased because samples taken during the hemoglobin production period of runs contained excess heme in the supernatant after they were spun down in a microcentrifuge. First, it was decided to reduce the concentration of heme in the base feed from 2.0 mg/mL to 0.5 mg/mL which resulted in brown cultures indicating minimal hemoglobin production. Then the concentration of heme in the base feed was decreased from 2.0 mg/mL to 1.5 mg/mL which resulted in the reddest (highest hemoglobin producing) culture yet. Also, at this heme concentration there was no excess heme in the supernatant of the samples. However, this run was repeated, the culture was brown at the end. This indicated that there was a problem other than the tetracycline concentration that was causing plasmid loss in the culture.

It was noticed that the glucose concentration remained above 0.05% (0.5 g/L) throughout most of the run for all of the red cultures and the glucose concentration remained below 0.05% throughout most of the run for all the brown cultures. Surprisingly, however, the maximum OD600 of the culture was not affected by the low glucose concentrations; the maximum OD600 of the red culture was 269 whereas the maximum OD600 of the brown culture was 285. FIG. 10 shows agitation graphs for a red run (A) and a brown run (B); in the runs, a base feed with a heme concentration of 1.5 mg/mL was used. The glucose concentration readings for the two runs are shown in yellow letters in FIG. 10. It was also noticed that runs where the glucose concentration remained above 0.05% (0.5 g/L) had smooth agitation graphs (FIG. 10A) and runs where the glucose concentration remained below 0.05% had noisy agitation graphs (FIG. 10B) during the first 36 hours of growth. As discussed above, the rate of agitation increases in order to aerate the culture more efficiently as the number of bacteria in the bioreactor increases. When the agitation speed reaches the maximum setting, pure oxygen is fed into the culture to maintain the dissolved oxygen level at the desired percentage. After 36 hours noise occurred in the agitation graphs but it was caused by the rapid fluctuations in dissolved oxygen that occur when pure oxygen is fed into the culture.

Glucose concentration also affects the plasmid concentration. In red runs (FIG. 10A) where the glucose concentration was normally above 0.05% and the first 36 hours of the agitation graph was smooth the plasmid concentration remained high (FIG. 11 lanes 3-5). In brown runs (FIG. 10B) where the glucose concentration was normally below 0.05% and the first 36 hours of the agitation graph was noisy the plasmid concentration drastically decreased (FIG. 11 lanes 7-9). With the developed glucose feed (FIG. 12 pink line) cultures were consistently grown to high cell densities but a high plasmid concentration in the cultures was not maintained.

To correct for this, the flow rate of the glucose feed was increased so that the glucose concentration of the culture would remain above 0.2% (2.0 g/L) for a majority of the run (FIG. 12 orange line). The flow rate of the new glucose feed was designed to account for the variable growth rates of different cultures. The old glucose feed (FIG. 12 pink line) failed to take into account the variable growth rates of different cultures; when cultures grew at a faster than expected rate the glucose concentration became too low (below 0.05%) and plasmid loss occurred. With the new glucose feed glucose concentrations above 0.2% for a majority of the run were maintained (FIG. 13). When the glucose concentration was maintained above 0.2%, the plasmid concentration of the culture decreased only slightly at the end of the run (data not shown) and the noise in the first 36 hours of the agitation graph disappeared (FIG. 13). With the new glucose feed (FIG. 12 orange line) a bioreactor culture was grown that produced hemoglobin at a concentration of 17.64 mg/mL (Table 6) as determined by hemoglobin assay performed.

TABLE 6

| | Hemoglobin | Hemoglobin | |
|---|---|---|---|
| | production per | production per | Maximum |
| Culture | milliliter | liter | OD600 |
| Bioreactor | 1.764 × 10−2 grams | 17.64 grams | 250 to 275 |
| Test Tube | 3.69 × 10−4 grams | 0.369 grams | 3 to 4 |

When hemoglobin assays were performed on cultures of this strain grown in test tubes, hemoglobin was produced at a concentration of 0.369 mg/mL (Table 6). Because cultures were denser in the bioreactor than in a test tube, the bioreactor culture produced 47.8 times more hemoglobin per mL than test tube cultures. A 1500 mL bioreactor culture produced 26.45 grams of hemoglobin (17.64 g/L) (Table 6). Since the performance of these experiments, it has been determined that the concentration may have been approximately 12 mg/mL (FIG. 19). One possible reason for this discrepancy is that the details of the hemoglobin assay in which the results are shown in Table 6 data had not fully been worked out but the details of the hemoglobin assay in the FIG. 19 data had been.

### Example 5

The purpose of this example was to scale up human hemoglobin production from BL21(DE3)/pHb0.0TM1hug cultures grown in test tubes to large scale (multiple liters) human hemoglobin production with cultures grown in a bioreactor. By developing a large scale human hemoglobin production method it was hoped to show that using E. coli that coexpress the P. shigelloides heme transport system genes and the human hemoglobin genes is an economical method of producing recombinant human hemoglobin for use as a blood substitute. Previous work by this lab suggested using a heme transport system to move heme into the cell created a large intracellular heme pool which allowed over 10 times more hemoglobin to be produced. The optimal glucose feed for production of high cell density cultures in a bioreactor was determined and then a strategy was developed to get the cells to produce large quantities of human hemoglobin. FIGS. 14 and 15 summarize the development of the glucose feed and the hemoglobin production strategy, respectively.

In the initial attempts at working out growth conditions, the E. coli strain was grown with a mixture of batch feeding (a large amount of glucose is added in a short period of time at set intervals) and constant-rate feeding (continuous addition of glucose at a set rate) (FIG. 14 item 1). With a batch/constant-rate glucose feed (FIG. 4), a culture was grown to a maximum OD600 of only 5. Even though the first glucose feeding strategy was a failure, two important lessons for developing the glucose feed were learned: (1) drastic swings in glucose concentration disrupt the growth of the bacteria. (2) the nutrient addition rate must match the bacterial growth rate.

A slightly modified version of a stepwise glucose addition technique was then used. (FIG. 5) (FIG. 14 item 2). The flow rate of a stepwise glucose feed is constant (which prevents drastic swings in glucose concentration) and increases as the bacterial growth rate increases. From the results of the stepwise glucose feed run (Table 1) it was discovered that the flow rate of the stepwise glucose feed (FIG. 5) was too high at the beginning and too low at the end. This led to the glucose concentration of the culture remaining at inhibitory levels during 80% of the stepwise glucose feed run and glucose starvation at the end of the run (Table 1). By using the stepwise glucose feed (FIG. 5), a culture was grown to a maximum OD600 of 53 (Table 1).

To avoid the guess work involved in correcting the stepwise glucose feed, the next glucose feed was based on the mass flow equation. By using the mass flow equation, how fast the glucose flow rate needed to increase to match the growth rate of the strain of E. coli may be calculated. Even though glucose accumulated to inhibitory levels (Table 2), a culture was grown that reached an OD600 of 93 (Table 2). To decrease the glucose accumulation and the resulting growth inhibitory acetic acid accumulation the flow rate of the predicted mass flow equation based nutrient feed was decreased (FIG. 7) so that the concentration of glucose in the culture would remain at 0.05% (0.5 g/L) or lower (FIG. 14 item 3). In addition to decreasing the flow rate of the mass flow equation based nutrient feed, two aspects of the growth strategy were changed: (1) the concentration of all of our feed solutions were increased to decrease the dilution of the culture from too much water volume (FIG. 14 item 3) (2) the size of the inoculum cultures were increased, to increase the number of bacteria using the glucose present in the culture, so that the glucose concentration of the culture would remain around 0.05% (FIG. 14 item 3). With these changes glucose accumulation was reduced but a maximum OD600 of only 116 was achieved (Table 3). It was observed that there was a large number of dead cells present at the bottom of pellets of late run samples which suggested at the time that the low glucose concentration led to the observed cell death which in turn resulted in a low maximum OD600.

To reduce the number of dead cells present in the culture at the end of a run, yeast extract was added to the media and glucose feed (FIG. 14 item 4). Yeast extract reduces accumulation of growth inhibitory acetic acid and acts as a second form of energy during the periods of glucose starvation that occur in a run where the glucose concentration is maintained around 0.5%. To counteract the basic waste products associated with the breakdown of yeast extract as an energy source, a 5.0 M phosphoric acid feed was added to the bioreactor setup. With a 1% yeast extract concentration in the starting media and a 0.97% yeast extract concentration in the glucose feed a culture was grown to a maximum OD600 of 255 (data not shown). However, when attempts were made to replicate the results, the culture reached a maximum OD600 of only 100 (data not shown). In an attempt to solve the inconsistent growth problem, the yeast extract concentration in the glucose feed was reduced to 0.485% and a slight improvement in the maximum OD600 (OD600 of 137 versus OD600s of 100 and 128.7) was seen.

It was then determined that the inconsistent growth was not due to the concentration of yeast extract in the glucose feed, but instead was a result of phosphate depletion. The culture that grew to a maximum OD600 of 255 had received numerous additions of phosphoric acid (FIG. 8) as a result of an improperly programmed pH controller. Once the pH controller was properly programmed, phosphoric acid was rarely added and a drastic decrease in the maximum OD600s of the yeast extract cultures occurred. To prevent phosphate depletion, a phosphate feed was added to the bioreactor setup (FIG. 14 item 5). With the phosphate feed and a modified version of the second mass flow equation based feed (FIG. 9 purple line), cultures were consistently grown to an OD600 around 265.

Prior to starting the development of the bioreactor hemoglobin production method, the test tube hemoglobin method was used to determine the optimal heme and IPTG concentration for this strain of E. coli. The increased cellular densities achieved with the bioreactor were taken into account to arrive at an IPTG concentration of 0.267 mg/mL for induction of the human hemoglobin genes (FIG. 15 item 1) and a heme concentration of 0.2 mg/mL for use in production of the human hemoglobin.

It was decided the best way to add the heme (which has to be dissolved in ammonium hydroxide) to the culture was to dissolve it in the 5.0 M ammonium hydroxide base feed at a concentration of 2.0 mg/mL (FIG. 15 item 1). By adding heme through the base feed, changing the pH of the culture was avoided and the rate of heme addition increased as the cellular density and acid waste products of the culture increased.

During the first 3 hemoglobin production runs, it was determined that induction did not slow down the growth rate of the culture and that the mid-exponential growth phase was the ideal time for induction of the human hemoglobin genes (FIG. 15 item 1). The most likely reason why hemoglobin production did not place a large metabolic burden on the cells was that the hemoglobin increased the effective dissolved oxygen concentration of the culture which allowed the culture to grow more efficiently.

After determining the initial hemoglobin production conditions heme feed was optimized. With a base feed that contained a heme concentration of 2.0 mg/mL there was excess heme in the supernatant of post-induction bioreactor samples when they were spun down in a microcentrifuge. To reduce the accumulation of excess heme, the heme concentration in the base feed was decreased to 1.5 mg/mL (FIG. 15 item 2).

It was next determined that plasmid loss was occurring frequently in our bioreactor runs. This resulted in inconsistent hemoglobin production levels. It was determined that this was due to two factors: (1) dilution of the tetracycline concentration as the volume of the culture increased and (2) low levels of glucose during crucial stages of growth. When the tetracycline concentration of the culture decreased there was less selection pressure for retention of high copy numbers of our plasmid which contains a tetracycline resistance gene. When the glucose concentration reached very low levels the bacteria reduced the number of copies of the plasmid to reduce their energy needs. To solve the tetracycline dilution problem a quantity of tetracycline was added at the time of induction (FIG. 15 item 3) that returned the culture to its original tetracycline concentration. To prevent periods where the glucose concentration was too low a higher flow rate glucose feed (FIG. 12) was designed to account for the variable growth rates of different cultures (FIG. 15 item 4). With these changes the plasmid concentration of the culture decreased only slightly at the end of the run (data not shown).

With the plasmid loss problem solved, cultures were grown that more consistently produced high amounts of hemoglobin (see Table 6 and FIG. 16). However, there was still room for improvement as indicated by comparing the hemoglobin production assay results of the small scale and large scale hemoglobin production methods. The bioreactor hemoglobin production method produced 32.35% less hemoglobin per OD600 unit than the test tube hemoglobin production method (Table 7). It should be noted once again that 17.64 g/L hemoglobin is much higher than the most recently reported hemoglobin concentration of 6.56 g/L. The higher concentration is the result of the hemoglobin assays not being sufficiently refined at that point in time. FIG. 18 is a photograph of culture from a representative hemoglobin run which demonstrates how red a hemoglobin producing culture becomes.

TABLE 7

| | | | | Potential hemo- |
|---|---|---|---|---|
| | | Hemo- | | globin production |
| | Hemo- | globin | | per liter based on |
| | globin | production | | test tube hemoglobin |
| | production | per OD600 | | production per |
| Culture | per liter | unit | OD600 | OD600 unit |
| Bioreactor | 17.64 grams | 0.069 grams | 250 to | 26.08 to 28.05 |
| | | | 275 | grams |
| Test Tube | 0.369 grams | 0.102 grams | 3 to 4 | — |

If the bioreactor hemoglobin production method produced 0.102 grams of hemoglobin per OD600 unit like the test tube hemoglobin production method (Table 7) 8.44 to 10.41 more grams of hemoglobin per liter would be able to be produced (based on the OD600 range of bioreactor cultures). The most likely cause of the reduced hemoglobin production per OD600 unit was the accumulation of acetic acid (as indicated by the odor of the culture). Acetic acid is one of the main inhibitory byproducts of E. coli growth and recombinant protein production in bioreactors. If acetic acid accumulation is reduced, there should be an increase in the quantity of hemoglobin produced per OD600 unit. In addition, the induction period on cultures grown in a tube is 16 h versus 10 h in the bioreactor, which may effect hemoglobin production.

It is believed there are numerous ways to increase hemoglobin production by either genetic manipulation or altering growth conditions. In terms of genetic modification, insertion of the alsS gene (which encodes the Bacillus subtilis protein acetolactate synthase) into the chromosome of BL21(DE3) would reduce acetic acid production. Excess pyruvate would be converted to acetolactate (instead of acetate) which would then be converted to acetoin which is 50 times less toxic than acetic acid. Another potentially beneficial genetic modification would be to delete the essential ssb gene (which encodes single strand binding protein) from the chromosome of BL21(DE3). The tetracycline resistance gene could then be replaced on the plasmid with the ssb gene. By using the ssb gene to force the bacteria to retain the plasmid, the low plasmid concentrations associated with tetracycline dilution and degradation would be eliminated. This would allow a high plasmid concentration to be consistently maintained which would increase the quantity of hemoglobin produced.

Altering the growth conditions is another method for increasing the quantity of hemoglobin produced. The growth temperature could be reduced to help stabilize the formation of hemoglobin precursors so that the quantity of soluble hemoglobin would increase. Lower temperatures could also slow down the growth of the culture during the induction period, which would lengthen the induction period so it more closely matches the induction period observed in a tube. Leucine, valine, and alanine (the amino acids that occur in the highest frequencies in the human hemoglobin protein) could be added to the culture at induction so that the bacteria do not have to synthesize those amino acids. It was assumed that fermentation media is optimal for hemoglobin production, but different media should be tried in order to optimize the production of hemoglobin and the functioning of the heme transport system. Finally, the IPTG concentration could be optimized so that the human hemoglobin genes are maximally induced.

With these changes it may be possible to greatly increase the quantity of human hemoglobin produced per OD600. With the current method, 17.64 grams of hemoglobin per liter may be produced (Table 6 and 7). If this method is improved so that bioreactor cultures produce the same quantity of hemoglobin per OD600 unit as the test tube cultures (0.102 g per OD600 unit), 15 grams of hemoglobin with 535 to 575 mL of culture may be produced. A single unit of packed red blood cells (450 to 475 mL) contains 57 to 81 grams of hemoglobin. Approximately 2 L to 3.1 L of culture would contain the amount of hemoglobin present in one unit of blood. Thus, the results of this example indicate that using E. coli that coexpress the P. shigelloides heme transport system genes and the human hemoglobin genes will be an economical method for producing recombinant human hemoglobin for use as a blood substitute.

### Example 6

What follows are additional modifications to the hemoglobin production procedure. This description includes many components of the procedure discussed thus far, as well as subsequent improvements that were made to the process.

Development of a fed-batch procedure for E. coli BL21(DE3)/pTHBHug.

The plasmid pTHBHug contains human hemoglobin genes with three mutations α(G15A)β(G16A/H116I) and the P. shigelloides heme transport genes from pHUG21. A fed-batch procedure was developed using a 3 L BioFlow 110 Modular Benchtop Fermentor (New Brunswick Scientific, Edison, N.J.) controlled by BioCommand Plus software. The inoculum was grown in DM-1 media (previously called “inoculum media”) and the bioreactor culture was grown in DM-4 media (previously called “fermentation media”) containing 0.2% yeast extract and 2 μg/mL tetracycline. Feed solutions were as follows: 1) An acid feed (5 M phosphoric acid) was used through the first 6 h of the run when the pH went above 6.9 and was replaced with a phosphate feed (2.4 M KH₂PO₄; 0.8 M K₂HPO₄) later in the run. Addition of phosphate late in the run was required for efficient glucose uptake and utilization; 2) A glucose feed (79.5% glucose, 0.5% yeast extract, 0.084 M MgSO₄-7H₂O, 0.154 g/L thiamin) was started 7 h into the run and continued throughout the run; 3) A base feed (5 M NH₄OH) was used until induction and then replaced with a heme feed (1.5 mg/mL heme in 5 M NH₄OH) in which the flow rate was pH-controlled. Hemoglobin genes, which are controlled by a tac promoter, were induced at around 30 h by the addition of 0.4 g of Isopropyl-®-D-thiogalactopyranoside (IPTG) to the bioreactor culture. Also added at induction was 1.87 mg of tetracycline (150 μL at 12.5 mg/mL). Dissolved oxygen was maintained at 40% and agitation was between 250 and 1200 rpm. A 1:2.5 dilution of Antifoam 204 (Sigma Chemical, St. Louis, Mo.) with water was used to control foaming; the flow rate for the antifoam was 0.5. The mass flow equation was used to estimate the glucose requirements of the strain.

FIG. 17 shows the flow rates of glucose and phosphate from a representative run, and the average growth curve of cultures from three runs. The average cell density (OD₆₀₀) of the cultures was 81.05+/−3.64 at induction and continued to increase after induction to an average OD600 of 280.9+/−15.08. The average final dry cell weight of the cultures was 83.64 g/L+/−3.22 g/L (FIG. 18). The dry cell weight values doubled from 2 h post induction to the end of the run, indicating induction of the hemoglobin genes did not inhibit the growth of the culture.

The glucose concentration of the culture as measured with an Accu-Chek Compact Plus glucose monitor (Roche, Mannheim, Germany) was maintained between 3.5 and 10.0 g/L during the first 28 h of the run. Glucose levels below 3.5 g/L during the first 28 h of the run correlated with plasmid loss in the cells (data not shown). Shortly after induction the glucose level was allowed to drop below 3.5 g/L. It was observed that if this drop in glucose level were not permitted to occur, the culture grew poorly the remainder of the run. This may have been due to the production of waste products such as acetic acid, which are detrimental to the health of the culture. The large error bar at 37.5 h may have been the result of one of the cultures having a glucose level of 0.4 g/L, which was much lower than that of the two other cultures reported in the graph. This low glucose level did not did not result in lower growth or hemoglobin production. By the end of the run, average glucose concentration was 0.25 g/L+/−0.23 g/L. The final volume of the culture was 1.557 L+/−0.049 L and the amount of heme added during the runs was 0.327 g+/−0.0175 g.

Hemoglobin Production During Bioreactor Runs.

To determine hemoglobin production in clarified cell lysates, 5.0 OD600 units of culture were centrifuged at 4° C., resuspended in 0.1 M Tris pH 7.5 that had been flushed with CO for 20 m, centrifuged again and the pellets frozen over night at −75° C. The cells were resuspended in CO-flushed 0.1 M Tris pH 8, and treated as previously described. Absorbance of clarified lysates was determined at 419 nm wavelength; absorbance of clarified lysates from the control strain BL21(DE3)/pCHug (pCHug contains only the heme transport genes) treated in the same fashion was subtracted from that value. To determine the concentration of hemoglobin, the Beer-Lambert equation was used with the molar extinction coefficient of carboxyhemoglobin. Hemoglobin in clarified lysates was not detectable at induction (30 h) but was detectable 2 h post induction (0.281 g/L+/−0.169 g/L). From 32 h to 36 h, soluble hemoglobin increased 14 fold to 3.95 g/L+/−1.24 g/L. The amount of hemoglobin at 40 h was 6.56 g/L+/−0.28 g/L. Looker et al., using a different strain of E con in which no heme transport system was present, produced 0.3805 g/L of hemoglobin in clarified lysates. The method described herein generated about 17-fold more hemoglobin per liter in clarified lysates. Hemoglobin from cells that had lysed late in the run was detectable at 39 h and reached an average of 0.334 g/L+/−0.301 g/L by the end of the run. More recently, runs have been done that have produced 8 g/L of hemoglobin.

FIG. 19B shows an immunoblot of hemoglobin from clarified lysates of BL21(DE3)/pTHBHug during a representative run. In agreement with FIG. 19A, no hemoglobin was detectable at induction (lane 2), very little hemoglobin was detected at 2 h post induction (32 h) (lane 3), and high amounts of hemoglobin were detected at 37.5 h and 40 h (lanes 4 and 5, respectively).

The above description indicates that production of recombinant hemoglobin in E. coli may be an economically viable method of obtaining therapeutic hemoglobin.

### Example 7

While the above examples describe certain embodiments of the present disclosure, certain variations of these embodiments are discussed below.

Different Media

Media other than fermentation media may be used for growth in the bioreactor vessel. In addition, the concentration of yeast extract in the bioreactor media and glucose feed will be increased to more closely match the yeast extract concentration in a test tube, where higher levels of hemoglobin are reported per cell density unit.

Oxygen Levels

While current procedures may use dissolved oxygen levels of 40%, concentrations above and below this level may be used.

Lowering Temperature of Culture after Induction

Fermentation literature has examples of situations where lowering the temperature has a positive impact on protein expression. Many labs working with hemoglobin expression actually use temperatures as low as 25° C. for expression. Temperatures lower than 36.5° C. may be beneficial for at least two reasons: to lengthen the time of induction by slowing the growth and to allow more stability of the hemoglobin that is made.

Different E. coli Strains and Plasmids

Strains other than BL21(DE3), such as TE5301 or TE5301 that has the Shigella dysenteriae heme receptor gene on the chromosome and the hemoglobin genes and P. shigelloides heme transport genes on a plasmid, or strains that have two different heme transport systems may be used. Plasmids containing the wild type hemoglobin genes and the P. shigelloides heme transport genes may also be used. A method called circular permutation may also be used in which components of the hemoglobin genes are rearranged.

Various Methods to Control Acetic Acid Production

As mentioned previously, accumulation of acetic acid may be detrimental to growth and protein production. Measuring acetic acid production during the runs may be beneficial.

Amino acids may be added to the fermentation media to reduce the formation of acetic acid. When the cells have to make these amino acids, the concentration of oxaloacetate, end product of the citric acid cycle, is lowered, which slows down the citric acid cycle. Acetic acid is made when the citric acid cycle cannot keep up with glycolysis (the initial breakdown of glucose to pyruvate). If some of the products of the citric acid cycle no longer need to be used to make these amino acids, then the citric acid cycle will have an easier time of keeping up with glycolysis.

Genes for enzymes that lead to lower acetic acid production may be moved into the hemoglobin producing strain. Pyruvate carboxylase, gene for aspartase, and gene for acetolactate synthase may be moved into the hemoglobin producing strain to reduce the production of acetic acid.

Adding Amino Acids to the Media that are Present in Hemoglobin in the Highest Amounts

There are about 4-5 amino acids which are present in large amounts in hemoglobin and may become limiting at some time during the induction process when the cells are having to make large quantities of hemoglobin. Adding this amino acid mix at induction or during induction may increase the hemoglobin yields.

Amount of Heme Per mL that May be Added to the Culture

Altering the concentration of heme used in the base feed may allow increased hemoglobin production.

Growth Time of the Culture

It has been shown that the growth time of the culture may only need to be 40 hours. It has also been found that three of the glucose feed steps at around 33 hours may need to be longer than shown in FIG. 12. It has also been found that the glucose feed may need to be monitored so glucose concentration in the culture remained between 3.5 g/L and 10 g/L during the first 30 hours of the run. The glucose concentration may need to drop well below 3.5 g/L at 32 hours into the run to about 1.0 g/L or the culture may stop growing.

Induction Time

It has been found that a time of induction of about 30 hours into the run at OD600 of about 80 may result in the healthiest growth and production of the largest amounts of hemoglobin. However, modifications can be made so induction occurs earlier in the run by decreasing the amount of IPTG initially added to turn on the hemoglobin genes and then adding additional IPTG at the normal 30 hour induction time. This alteration would involve starting the heme/base feed earlier so that it precedes the earlier induction time by several hours. In addition, glycerol can be added to the glucose feed at induction which can slow down growth sufficiently to increase induction time and allow more time for expression of the hemoglobin genes.

pH of the Culture

The pH of the culture can be altered so that the optimal pH for hemoglobin expression can be found. It is possible that the currently used pH is too low for optimal growth and hemoglobin production.

Therefore, the present invention is well adapted to attain the ends and advantages mentioned as well as those that are inherent therein. While numerous changes may be made by those skilled in the art, such changes are encompassed within the spirit of this invention.

What is claimed is:

1. A method for producing hemoglobin in a bioreactor comprising:
providing a bioreactor containing a strain of E. coli comprising hemoglobin producing genes and heme transport genes on a one plasmid system;
introducing a carbon source into the bioreactor;
growing a culture of the E. coli strain;
inducing the hemoglobin producing genes; and
allowing the E. coli to produce hemoglobin.

2. The method of claim 1, wherein the culture is grown on a defined media or a complex media.

3. The method of claim 1, wherein the carbon source comprises a glucose feed or a glycerol feed.

4. The method of claim 1, wherein the carbon source comprises a glucose feed and a yeast extract.

5. The method of claim 1, wherein the carbon source comprise a glucose feed that is introduced into the bioreactor at a rate calculated by using a mass flow equation.

6. The method of claim 1, further comprising introducing oxygen, base, and acid feeds into the bioreactor.

7. The method of claim 6, further comprising introducing a phosphate feed into the bioreactor.

8. The method of claim 7, wherein the phosphate feed replaces the acid feed.

9. The method of claim 7, wherein the phosphate feed is introduced into the bioreactor once the culture reaches an OD600 of 90.

10. The method of claim 7, further comprising introducing a heme feed into the bioreactor.

11. The method of claim 10, wherein the amount of heme introduced into the bioreactor is at least 0.21 mg/mL of the culture.

12. The method of claim 1, wherein the hemoglobin producing genes are induced when the culture reaches an OD600 of 80.

13. A method for producing hemoglobin in a bioreactor comprising:
providing a bioreactor containing a strain of E. coli comprising hemoglobin producing genes and heme transport genes on a one plasmid system;
introducing oxygen, base, acid, and glucose feeds into the bioreactor;
introducing a phosphate feed into the bioreactor;
growing a culture of the E. coli strain;
inducing the hemoglobin producing genes; and
allowing the E. coli to produce hemoglobin.

14. The method of claim 13, wherein the glucose feed is introduced into the bioreactor at a rate calculated by using a mass flow equation.

15. The method of claim 13, wherein the phosphate feed is introduced into the bioreactor once the culture reaches an OD600 of 90.

16. The method of claim 13, further comprising introducing a heme feed into the bioreactor.

17. The method of claim 16, wherein the heme feed comprises ammonium hydroxide.

18. The method of claim 13, wherein the hemoglobin producing genes are induced when the culture reaches an OD600 of 80.

19. A method for producing hemoglobin in a bioreactor comprising:
providing a bioreactor containing a strain of E. coli comprising hemoglobin producing genes and heme transport genes on a one plasmid system;
introducing a glucose feed into the bioreactor;
introducing a heme feed comprising ammonium hydroxide into the bioreactor;
growing a culture of the E. coli strain;
inducing the hemoglobin producing genes; and
allowing the E. coli to produce hemoglobin.

20. The method of claim 19, wherein:
the glucose feed is introduced into the bioreactor at a flow rate calculated by using a mass flow equation;
the amount of heme introduced into the bioreactor is at least 0.21 mg/mL of the culture;
the amount of ammonium hydroxide introduced into the bioreactor is sufficient to maintain a pH of about 6.8 in the bioreactor; and
the hemoglobin producing genes are induced when the culture reaches an OD600 of 80.
